(19) United States
(12) Patent Application Publication
(10) Pub. No.: US 2014/0302491 A1
(43) Pub. Date: Oct. 9, 2014

(54) Ex Vivo Culture, Proliferation and Expansion of Primary Tissue Organoids

(71) Applicant: The Board of Trustees of the Leland Stanford Junior University, Palo Alto, CA (US)

(72) Inventors: Lincoln Nadauld, Los Altos, CA (US); Calvin Jay Kuo, Palo Alto, CA (US)

(21) Appl. No.: 14/354,390

(22) PCT Filed: Oct. 29, 2012
(86) PCT No.: PCT/US12/62454

§ 371 Date: Apr. 25, 2014

Related U.S. Application Data
(60) Provisional application No. 61/552,932, filed on Oct. 28, 2011.

(51) Int. Cl. G01N 33/50

(52) U.S. Cl. CPC G01N 33/5017; G01N 33/5008; G01N 33/5011
USPC 435/5; 435/325; 435/29

(57) ABSTRACT

Culture systems and methods for long term culture of mammalian tissues are provided. Tissues include but are not limited to lung alveolar tissue, stomach tissue, pancreas tissue, bladder tissue, liver tissue, and kidney tissue. Cultures are initiated with fragments of mammalian tissue, which are then cultured embedded in a gel substrate that provides an air-liquid interface. Cultured explants of the invention can be continuously grown in culture for a year or more, while maintaining features of the tissue including prolonged tissue expansion with proliferation, multilineage differentiation, and recapitulation of cellular and tissue ultrastructure.

### CROSS REFERENCE TO RELATED APPLICATIONS

Pursuant to 35 U.S.C. §119 (e), this application claims priority to the filing date of the U.S. Provisional Patent Application Ser. No. 61/552,932 filed Oct. 28, 2012; the disclosure of which is herein incorporated by reference.

### GOVERNMENT RIGHTS

This invention was made with Government support under contract DK085720 awarded by the National Institutes of Health. The Government has certain rights in this invention.

### BACKGROUND

A significant impediment to tissue engineering, disease modeling, and drug discovery has been a notable lack of in vitro culture systems that provide for the growth of tissue explants for more than about 10 days. What is needed is a single primary 3D “organoid” culture method that is broadly applicable to numerous explanted tissues and that provides for long-term proliferation, multi-lineage differentiation, and explant characteristics that recapitulate the in vivo cellular and tissue ultrastructure.

### RELEVANT LITERATURE

A number of publications discuss various methods for culturing different cell types including intestinal epithelial cells. Toda et al in Cell Biology: A Laboratory Handbook, Vol. 1, Chapter 50, describe thyroid tissue-organotypic culture using an approach for overcoming the disadvantages of conventional organ culture. The teachings of the culture methods of Toda et al. are hereby incorporated by reference. Establishment of a long-term culture system for rat colon epithelial cells is described by Bartsch et al. in In Vitro Cell Dev Biol Anim. 2004 September-October; 40(8-9):278-84. Panja et al in Lab Invest. 2000 September; 80(9):1473-5 describe a method for the establishment of a pure population of nontransformed human intestinal primary epithelial cell (HIPEC) lines in long term culture. A method for long-term culture of primary small intestinal epithelial cells (IEC) from suckling mice is described by Macartney et al in J. Virol. 2000 June; 74(12):5597-603. Baten et al discuss methods for long-term culture of normal human colonic epithelial cells in vitro. Sambuy; De Angelis I in Cell Differ. 1986 September; 19(2):139-47 describe formation of organoid structures and extracellular matrix production in an intestinal epithelial cell line during long-term in vitro culture. U.S. application Ser. No. 12/545,755 and Ootani et al. in Nat. Med. 2009 June; 15(6):701-6 describe a method for long term culture of mammalian intestinal cells and the production of intestinal organoids by this culture method. Yamaya et al. in Am J. Physiol. 1992 June; 262(6 Pt 1):L713-24, Dobbs et al. Am J. Physiol. 1997 August; 273(2 Pt 1):L347-54, and Fulcher et al. in Methods Mol. Med. 2005; 107:183-206 describe the differentiation of tracheal cells, alveolar type II cells, and airway epithelial cells, respectively, in culture.

### SUMMARY OF THE INVENTION

Culture systems and methods for long term culture of mammalian tissues are provided. Tissues include but are not limited to lung alveolar tissue, stomach tissue, pancreas tissue, bladder tissue, liver tissue, and kidney tissue. Cultures are initiated with fragments of mammalian tissue (“explants”), which are then cultured embedded in a gel substrate that provides an air-liquid interface. Cultured explants of the invention can be continuously grown in culture for extended periods of time, for example for 1 month or more, e.g. for one year or more. Mammalian tissues explants cultured by the methods of the invention recapitulate features of tissue growth in vivo. Features include, without limitation, prolonged tissue expansion with proliferation, multilineage differentiation, and recapitulation of cellular and tissue ultrastructure, including epithelial tissues, submucosal tissues, and stromal environments, While the culture system provides for growth of the varied cells found in normal mammalian tissues, the cultures are also useful in the generation of cells for selection, to provide purified population or enriched populations of a single lineage for any given tissue, including tissue-specific stem cells. Organoids cultured by these methods find use in many applications such as tissue engineering, disease modeling, and drug discovery.

The cultured cells may be experimentally modified prior, or during the culture period. In some embodiments, the cells are modified by exposure to viral or bacterial pathogens. In other embodiments the cells are modified by altering patterns of gene expression, e.g. by providing reprogramming factors to induce pluripotency or otherwise alter differentiation potential; or by introducing cancer drivers that provide for oncogenic transformation of cells into carcinomas, e.g. nucleic acids encoding Kras^{G12D}; nucleic acids that suppress expression of APC, p53, or Smad4; etc. The experimentally modified cells are useful for investigation of the effects of therapeutic agents for anti-viral or anti-bacterial activity; for tumor therapy, for effects on differentiation, and the like. For example, the effect of a gain or loss of gene activity on the ability of cells to form an explant culture may be determined, or on the ability to undergo tumor transformation.

In another aspect of the invention, a method is provided for in vitro screening for agents for their effect on cells of different tissues, including processes of cancer initiation and treatment, and including the use of experimentally modified cultures described above. Tissue explants cultured by the methods described herein are exposed to candidate agents. Agents of interest include pharmaceutical agents, e.g. small molecules, antibodies, peptides, etc., and genetic agents, e.g. antisense, RNAi, expressible coding sequences, and the like, e.g. expressible coding sequences for candidate tumor suppressors, candidate oncogenes, and the like. In some embodiments, the effect on stem cells is determined. In other embodiments the effect of transformation or growth of tumor cells is determined, for example where agents may include, without limitation, chemotherapy, monoclonal antibodies or other protein-based agents, radiation/radiation sensitizers, cDNA, siRNA, shRNA, small molecules, and the like. Agents active on tissue-specific stem cells are detected by change in growth of the tissue explants and by the presence of multilineage differentiation markers indicative of the tissue-specific stem cell. In addition, active agents are detected by analyzing tissue explants for long-term reconstitutive activity. Methods are also provided for using the explant culture to screen for agents that modulate tissue function. In some embodiments, the methods find use in identify new agents for the treatment of disease. In some embodiments, the methods find use in screening a known therapeutic agent to determine if that agent will prevent or treat disease in an individual from which an explant has been prepared. In other words, the screen is used to predict the responsiveness of an individual to therapy, e.g. an anti-viral therapy, an anti-bacterial therapy, a cancer therapy (e.g. an anti-tumorigenic or anti-tumoral therapy), etc.

Methods are provided for screening cells in a population, e.g. a complex population of multiple cells types, a population of purified cells isolated from a complex population by sorting, culture, etc., and the like, for the presence of cells having stem cell potential. This method entails co-culture of detectably labeled candidate cells with the tissue explant of the invention. Candidate cells with stem cell potential are detected by an increase in growth of the cultured explant above basal levels and colocalization of multilineage differentiation markers indicative of the presence of tissue-specific stem cells with the labeled candidate cells. Stem cell characteristics of candidate cells co-cultured with explants are further assayed by determining long-term reconstitutive activity, via in vivo transplantation, etc.

In another aspect of the invention, a method is provided for in vitro screening of agents for cytotoxicity to different tissues, by screening for toxicity to explant cultures of the invention. In yet another embodiment, a method is provided to assess drug absorption by different tissues, by assessing absorption of a drug by explant cultures of the invention.

### BRIEF DESCRIPTION OF THE DRAWINGS

FIG. 1. Primary intestinal organoids exhibit long-term differentiation, proliferation and intestinal stem cells. A. Schematic of organoids within a collagen gel and air-liquid interface. B, C. Sustained growth of neonatal mouse colon organoids at d8 and d357. D-F. Proliferation, enterocytes, and goblet cells in colon cultures, d92 and d357. G. Multilineage differentiation in colon organoids, d120-d223. H. Lgr5+ intestinal stem cells. Fluorescent in situ. (Ootani, A., et al. (2009) Sustained in vitro intestinal epithelial culture within a Wnt-dependent stem cell niche. Nat Med 15, 701-706).

FIG. 2. Adenovirus Cre activates a APC/KRas/p53 3-oncogene module (AKP) in APCflox/flox; LSL KRasG12D; p53flox/flox colon and lung organoids. (A-C). Neonatal colon organoids. (D-F). Adult colon organoids. (G-H). Adult lung organoids. In all cases, Ad Cre or the control Ad Fc was added at d0 at plating in ALI culture and harvested at d21. Marked dysplasia is seen with 3-gene AKP but not the 1-gene APC (“A”). H&E, 10×.

FIG. 3. A 4-gene AKPS module in primay colon organoids (A). Adeno GFP infection. (B-D) Simultaneous retrovirus GFP/RFP yields quantitative co-infection. (E-M) An APC/KRas/p53/Smad4 (AKPS) 4-gene module. APC-null (APCflox/flox; villin-CreER+tamoxifen) cultures were not infected (E-H) or infected with retroviruses encoding KRasG12D, p53shRNA and Smad4 shRNA. (E-L). Effective KRasG12D expression, p53 knockdown and IRES-GFP expression in E-Cad+ epithelium. (M) FACS sorting/qPCR of AKPS EpCAM+/GFP+(i.e. retro-infected epithelium) reveals Smad4 knockdown.

FIG. 4. Oncogene modules of different complexity in primary colon organoids. A. APC loss (1-gene) leads to hyperproliferation without significant dysplasia. APC^{flox/flox}; villin-CreER colon organoids were treated with tamoxifen in vitro, d20. B-D. 2-gene modules APC^{-/-}/KRas^{G12D} (AK), APC^{-/-}/p53 shRNA (AP) or APC^{-/-}/Smad4 shRNA (AS) with the second non-APC hit delivered by retrovirus do not induce dysplasia. E-I. The 4-gene module (AKPS) exhibits pronounced dysplasia exceeding 1-, 2- or 3-gene modules. Mean+/−SE. P value: *: A vs AKP or AKP* P<=0.0072; **: A vs AKPS P<0.0001.

FIG. 5. Serial passage and in vivo transplantation of colon 4-gene AKPS organoids. (A, B). Serial passage in ALI culture reveals extremely robust growth. (C, D). AKPS cells grow as solid tumor masses in ALI upon serial passage. (E, F). Growth and focus formation of AKPS cells on plastic after serial ALI culture. (G, H). In vivo transplantation of AKPS (50,000 cells) subcutaneously into NSG mice vs no take with APC-null (“A”). (H). H&E AKPS tumor, d30.

FIG. 6. Histologic transformation of primary gastric organoids. (A-F). Wild-type gastric organoids in air-liquid interface culture grow as epithelial spheres (A,D), express differentiation markers (B,C) and are readily infected by adenovirus and retrovirus without microinjection (E, F). (G-L). Gastric organoids from adult KRas^{G12D}; p53^{flox/flox} mice (G, J) exhibit marked growth induction and pronounced dysplasia upon adenovirus Cre infection. Day 30 is depicted.

FIG. 7. Efficient transformation of primary lung organoids. (A-F). WT lung organoids in ALI culture grow with bronchiolar and alveolar architecture, express surfactant protein-B, and are easily infected with adenovirus. (G, H). Robust retroviral infection of lung organoids with retro KRas^{G12D}+retro p53 shRNA IRES GFP is associated with marked dysplasia, d28.

FIG. 8. Synergistic transformation of lung alveolar organoids by KRas^{G12D} and p53. (A-I). Primary plating of LSL KRas^{G12D}, p53^{flox/flox} of KRas^{G12D}; p53^{flox/flox} lung organoids +/− Ad Cre infection indicates more prominent growth (A-D) and histologic transformation (E-I) with combined KRas^{G12D} and p53 loss. (J-N) Serial replating assay demonstrates synergistic growth induction by combined KRas^{G12D} and p53 loss.

FIG. 9. Robust and reproducible organoid growth and retroviral infection in 96w transwells. A. Lung KP organoids were disaggregated and freshly passaged into 96 well transwells; bottom panel is enlargement showing robust sphere formation. (B, C) Cells from Colon AKPS organoids, or lung, gastric, pancreas KP organoids form secondary organoids upon replating in 96 well transwells, d2. Cell # detected at day 2 by CellTiter-Glo (n=6,+/− standard error). D. Efficient infection by retrovirus GFP upon fresh replating into 96 well transwells. Empty retrovirus showed no GFP signal.

FIG. 10. Neonatal kidney, bladder or lung were dissected, minced and plated in a collagen matrix with an air-liquid interface. They were then treated adenoviral-Fc (AdFc) or Adenovirus CreGFP to induce deletion of p53, and activation of Kras. The images above correspond to light microscope images (LM), fluorescent images of GFP confirming adenovirus infection, and Hematoxylin/Eosin staining to confirm viable tissue. These images confirm that these tissues grow viably in an air-liquid interface and are genetically tractable through introduction of virus.

FIG. 11. Neonatal kidney was dissected, minced and plated in a collagen matrix with an air-liquid interface. It was then treated adenoviral-Fc (AdFc) or Adenovirus CreGFP to induce deletion of p53, or activation of Kras. The images above correspond to light microscope images (LM), and fluorescent images of GFP confirming adenovirus infection. These images confirm that these tissues grow viably in an air-liquid interface and are genetically tractable through introduction of virus.

FIG. 12. Neonatal murine kidney with the indicated genotypes (P53fl/fl, KRasG12DLSL), was dissected, minced, and plated in a collagen matrix with an air-liquid interface. It was then treated with adenoviral-Fc (AdFc) or Adenovirus CreGFP to induce deletion of p53, or activation of KRasG12D, or both. The images above demonstrate kidney spheres growing in the collagen matrix on d1 or d14 after preparation. Spheres were also sectioned and stained by H&E to reveal the dysplastic (P53 or KRas+AdCre) or transformed (P53&Kras+AdCre) renal epithelium.

FIG. 13. Neonatal murine bladder with the P53fl/fl; KRasG12DLSL genotype was dissected, minced, and plated in a collagen matrix with an air-liquid interface. It was then treated with adenoviral-Fc (AdFc) or Adenovirus CreGFP to induce deletion of p53 and activation of KRasG12D. The images above demonstrate bladder spheres growing in the collagen matrix on d1 or d14 after preparation. Spheres were also sectioned and stained by H&E to reveal the transformed (P53&Kras+AdCre) bladder epithelium.

FIG. 14. Primary mouse pancreatic organoid culture. Brightfield images, GFP fluorescence after infection by adenovirus Cre-GFP, and immunofuorescence for the markers E-Cadherin, Pdx1, PCNA and insulin are depicted. Day 10 of culture is indicated.

FIG. 15. Oncogenic transformation of pancreatic organoid culture from LSL KRas^{G12D}; p53^{flox/flox} mice, with and without adenovirus Cre-GFP infection. Cre expression is associated with Kras activation and p53 deletion and increased growth as well as histologic transformation.

FIG. 16. Primary adult human colon organoid air-liquid interface culture. Day 10 of culture is depicted.

### DEFINITIONS

In the description that follows, a number of terms conventionally used in the field of cell culture are utilized extensively. In order to provide a clear and consistent understanding of the specification and claims, and the scope to be given to such terms, the following definitions are provided.

The term “cell culture” or “culture” means the maintenance of cells in an artificial, in vitro environment. It is to be understood, however, that the term “cell culture” is a generic term and may be used to encompass the cultivation not only of individual cells, but also of tissues or organs.

The term “culture system” is used herein to refer to the culture conditions in which the subject explants are grown that promote prolonged tissue expansion with proliferation, multilineage differentiation and recapitulation of cellular and tissue ultrastructure.

“Gel substrate”, as used herein has the conventional meaning of a semi-solid extracellular matrix. Gel described here in includes without limitations, collagen gel, matrigel, extracellular matrix proteins, fibronectin, collagen in various combinations with one or more of laminin, entactin (nidogen), fibronectin, and heparin sulfate; human placental extracellular matrix.

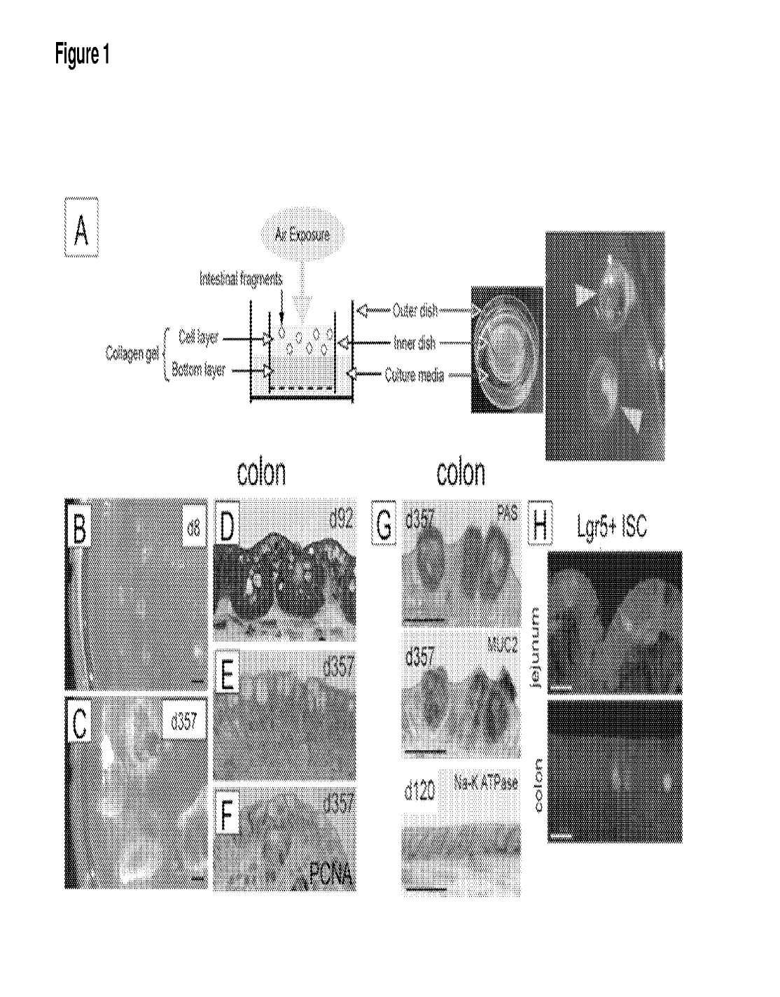

An “air-liquid interface” is the interface to which the intestinal cells are exposed to in the cultures described herein. The primary tissue may be mixed with a gel solution which is then poured over a layer of gel formed in a container with a lower semi-permeable support, e.g. a membrane. This container is placed in an outer container that contains the medium such that the gel containing the tissue in not submerged in the medium. The primary tissue is exposed to air from the top and to liquid medium from the bottom (FIG. 1A).

By “container” is meant a glass, plastic, or metal vessel that can provide an aseptic environment for culturing cells.

The term “explant” is used herein to mean a piece of tissue and the cells thereof originating from mammalian tissue that is cultured in vitro, for example according to the methods of the invention. The mammalian tissue from which the explant is derived may obtained from an individual, i.e. a primary explant, or it may be obtained in vitro, e.g. by differentiation of induced pluripotent stem cells.

The term “organoid” is used herein to mean a 3-dimensional growth of mammalian cells in culture that retains characteristics of the tissue in vivo, e.g. prolonged tissue expansion with proliferation, multilineage differentiation, recapitulation of cellular and tissue ultrastructure, etc. A primary organoid is an organoid that is cultured from an explant, i.e. a cultured explant. A secondary organoid is an organoid that is cultured from a subset of cells of a primary organoid, i.e. the primary organoid is fragmented, e.g. by mechanical or chemical means, and the fragments are replated and cultured. A tertiary organoid is an organoid that is cultured from a secondary organoid, etc.

The phrase “mammalian cells” means cells originating from mammalian tissue. Typically, in the methods of the invention pieces of tissue are obtained surgically and minced to a size less than about 1 mm³, and may be less than about 0.5 mm³, or less than about 0.1 mm³. “Mammalian” used herein includes human, equine, bovine, porcine, canine, feline, rodent, e.g. mice, rats, hamster, primate, etc. “Mammalian tissue cells” and “primary cells” have been used interchangeably.

“Stem cell” is used herein to refer to a mammalian cell that has the ability both to self-renew and to generate differentiated progeny (see Morrison et al. (1997) Cell 88:287-298). Generally, stem cells also have one or more of the following properties: an ability to undergo asymmetric replication, i.e. where the two daughter cells after division can have different phenotypes; extensive self-renewal capacity; and capacity for existence in a mitotically quiescent form.

Stem cells may be characterized by both the presence of markers associated with specific epitopes identified by antibodies and the absence of certain markers as identified by the lack of binding of specific antibodies. Stem cells may also be identified by functional assays both in vitro and in vivo, particularly assays relating to the ability of stem cells to give rise to multiple differentiated progeny.

By “pluripotent stem cell” or “pluripotent cell” it is meant a cell that has the ability to differentiate into all types of cells in an organism. Pluripotent cells are capable of forming teratomas and of contributing to ectoderm, mesoderm, or endoderm tissues in a living organism. Examples of pluripotent stem cells are embryonic stem (ES) cells, embryonic germ stem (EG) cells, and induced pluripotent stem (iPS) cells.

By “embryonic stem cell” or “ES cell” it is meant a cell that a) can self-renew, b) can differentiate to produce all types of cells in an organism, and c) is derived from the inner cell mass of the blastula of a developing organism. ES cells can be cultured over a long period of time while maintaining the ability to differentiate into all types of cells in an organism. ES cells are considered to be undifferentiated when they have not committed to a specific differentiation lineage. In culture, ES cells typically grow as flat colonies with large nucleo-cytoplasmic ratios, defined borders and prominent nuclei. In addition, ES cells express SSEA-3, SSEA-4, TRA-1-60, TRA-1-81, and Alkaline Phosphatase, but not SSEA-1. Examples of methods of generating and characterizing ES cells may be found in, for example, U.S. Pat. No. 7,029,913, U.S. Pat. No. 5,843,780, and U.S. Pat. No. 6,200,806, the disclosures of which are incorporated herein by reference.

By “embryonic germ stem cell”, embryonic germ cell” or “EG cell” it is meant a cell that a) can self-renew, b) can differentiate to produce all types of cells in an organism, and c) is derived from germ cells and germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, the disclosures of which are incorporated herein by reference.

By “induced pluripotent stem cell” or “iPS cell” it is meant a cell that a) can self-renew, b) can differentiate to produce all types of cells in an organism, and c) is derived from a somatic cell. iPS cells have an ES cell-like morphology, growing as flat colonies with large nucleo-cytoplasmic ratios, defined borders and prominent nuclei. In addition, iPS cells express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPS cells may be found in, for example, Application Nos. US20090047263, US20090068742, US20090191159, US20090227032, US20090246875, and US20090304646, the disclosures of which are incorporated herein by reference.

“Lineage-committed stem cells” is used herein to refer to multipotent stem cells that give rise to cells of specific lineage, e.g. mesodermal stem cells (see, e.g. Reyes et al. (2001) Blood 98:2615-2625; Eisenberg & Bader (1996) Circ Res. 78(2):205-16; etc.)

“Tissue-specific stem cells” is used herein to refer to multipotent stem cells that reside in a particular tissue and are capable of clonal regeneration of cells of the tissue in which they reside, for example the ability of hematopoietic stem cells to reconstitute all hematopoietic lineages, or the ability of neuronal stem cells to reconstitute all neuronal/glial lineages. “Progenitor cells” differ from tissue-specific stem cells in that they typically do not have the extensive self-renewal capacity, and often can only regenerate a subset of the lineages in the tissue from which they derive, for example only lymphoid or erythroid lineages in a hematopoietic setting, or only neurons or glia in the nervous system.

Culture conditions of interest provide an environment permissive for differentiation, in which stem cells will proliferate, differentiate, or mature in vitro. Such conditions may also be referred to as “differentiative conditions”. Features of the environment include the medium in which the cells are cultured, any growth factors or differentiation-inducing factors that may be present, and a supporting structure (such as a substrate on a solid surface) if present. Differentiation may be initiated by formation of embryoid bodies (EB), or similar structures. For example, EB can result from overgrowth of a donor cell culture, or by culturing ES cells in suspension in culture vessels having a substrate with low adhesion properties.

The term “multi-lineage differentiation markers” means differentiation markers characteristic of different cell-types. These differentiation markers can be detected by using an affinity reagent, e.g. antibody specific to the marker, by using chemicals that specifically stain a cell type, etc as known in the art.

“Ultrastructure” refers to the three-dimensional structure of a cell or tissue observed in vivo. For example, the ultrastructure of a cell may be its polarity or its morphology in vivo, while the ultrastructure of a tissue would be the arrangement of different cell types relative to one another within a tissue.

The term “candidate cells” refers to any type of cell that can be placed in co-culture with the tissue explants described herein. Candidate cells include without limitations, mixed cell populations, ES cells and progeny thereof, e.g. embryoid bodies, embryoid-like bodies, embryonic germ cells.

The term “candidate agent” means any oligonucleotide, polynucleotide, siRNA, shRNA, gene, gene product, peptide, antibody, small molecule or pharmacological compound that is introduced to an explant culture and the cells thereof as described herein to assay for its effect on the explants.

The term “contacting” refers to the placing of candidate cells or candidate agents into the explant culture as described herein. Contacting also encompasses co-culture of candidate cells with tissue explants for at least 1 hour, or more than 2 hrs or more than 4 hrs in culture medium prior to placing the tissue explants in a semi-permeable substrate. Alternatively, contacting refers to injection of candidate cells into the explant, e.g. into the lumen of an explant.

“Screening” refers to the process of either co-culturing candidate cells with or adding candidate agents to the explant culture described herein and assessing the effect of the candidate cells or candidate agents on the explant. The effect may be assessed by assessing any convenient parameter, e.g. the growth rate of the explant, the presence of multilineage differentiation markers indicative of stem cells, etc. The effect of candidate cells or candidate agents on the explant can be further evaluated by assaying the explant for long-term reconstitutive activity by serial in vitro passage, as well as by in vivo transplantation.

The terms “transformed” or “oncogenically transformed” as used herein refers to the process by which normal cells become tumorigenic, i.e. cancer cells.

The term “cancer drivers” as used herein refers to genomic aberrations or other cellular modifications that promote the transformation of cells. Examples of cancer drivers include loss-of-function tumor suppressor mutations, or agents that suppress expression or activity of tumor suppressors, and gain-of-function oncogene mutations, or agents that promote expression or activity of oncogenes. Cancer drivers may act alone and/or in combination, e.g. synergistically, to promote transformation. Combinations of cancer drivers that act together to more effectively promote tumorigenesis are referred to herein as “cancer driver modules”.

### DESCRIPTION OF THE SPECIFIC EMBODIMENTS

Culture systems and methods are provided for long term culture of various mammalian tissues, including but not limited to lung alveolar tissue, stomach tissue, pancreas tissue, bladder tissue, liver tissue, and kidney tissue. By long term culture, it is meant continuous growth of the explant for extended periods of time, e.g. for 15 days or more, for 1 month or more, for 2 months or more, for 3 months or more, for 6 months or more, or up to a year, or more. By continuous growth, it is meant sustained viability, organization, and functionality of the tissue. For example, unless experimentally modified, proliferating cells in a tissue explant that undergoes continuous growth in the culture systems of the present application will continue to proliferate at their natural rate, while non-proliferative, e.g. differentiated, cells in the tissue explant will remain in a quiescent state. Because of this, explants cultured by the subject methods are referred to as “organoids”.

In some embodiments, tissue, i.e. primary tissue, is obtained from a mammalian organ. The tissue may be from any mammalian species, e.g. human, equine, bovine, porcine, canine, feline, rodent, e.g. mice, rats, hamster, primate, etc The mammal may be of any age, e.g. a fetus, neonate, juvenile, adult. The following are some non-limiting examples of tissues that may be obtained for the purposes of preparing organoids:

Lung Alveolar Tissue.

Lung alveolar organoids are organoids derived from lung alveolar tissue. The lung alveoli are where the gas exchange of carbon dioxide and oxygen takes place. As such, lung alveolar tissue comprises a unique tissue structure and cellular composition relative to other tissues of the respiratory system. Alveolar tissue comprises two cellular layers, an alveolar epithelium and a capillary endothelium, which are separated by a thin interstitial space. There are two types of cells in the alveolar epithelium: type I (Squamous Alveolar) cells and type II (Great Alveolar) cells. Type I cells are squamous epithelial cells that have long cytoplasmic extensions which spread out thinly along the alveolar walls. They are identifiable by their unique shape and their expression of T1 alpha. Type II cells are cuboidal epithelial cells and are responsible for producing surfactant, a phospholipid which lines the alveoli and serves to differentially reduce surface tension at different volumes, contributing to alveolar stability. They can be identified by their cuboidal shape and their expression of SP-C and CC10 See, e.g., Meneghetti et al. Diseases that can be modeled in vitro using lung alveolar organoids include emphysema, in which lung elasticity is lost because the elastin in the walls of the alveoli is broken down by an imbalance between the production of neutrophil elastase (elevated by cigarette smoke) and alpha-1-antitrypsin (the activity varies due to genetics or reaction of a critical methionine residue with toxins including cigarette smoke). Other diseases include lung cancers, e.g. squamous cell carcinoma, adenocarcinoma, large-cell carcinoma; fibrotic disease; and pneumonia e.g. due to vasculitis, collagen vascular disease (e.g. Sjogren's syndrome), granulomatous disease (e.g. Sarcoidosis), or viral, bacterial, or fungal infection.

Stomach Tissue.

Stomach organoids are organoids derived from stomach, or gastric, tissue. The stomach is a muscular, hollow, dilated part of the alimentary canal. It comprises a mucosal layer comprising mucosal epithelium and lamina propria; which is surrounded by a submucosal layer comprising loose connective tissue; which is surrounded by a muscularis layer comprising several thick layers of muscle. The mucosal epithelium is comprised of four major types of secretory epithelial cells: mucous cells, which secrete an alkaline mucus that protects the epithelium against shear stress and acid; parietal cells, which secrete hydrochloric acid; chief cells (also called “peptic cells”) which secrete the zymogen pepsinogen; and G cells, which secrete the hormone gastrin. The epithelium is folded into thousands of tiny pits, called gastric pits, at the base of which are gastric glands; the mucous cells reside at the neck of the pits, while the chief cells and parietal cells residue at the base of the pits, in the glandular zone. Other markers of terminal gastric epithelial differentiation include H+/K+ atpase and mucin (MUC5A).

Stomach tissue also comprises a stomach-specific stem cell, a villin⁺Lgr⁵⁺ cell which is able to give rise to all gastric cell lineages. This stem cell is described in greater detail in Qiao XT and Gumucio DL. Current molecular markers for gastric progenitor cells and gastric cancer stem cells. J. Gastroenterol. 2011 July; 46(7):855-65, the disclosure of which is incorporated herein by reference.

Diseases that can be modeled in vitro with stomach organoids include, without limitation, stomach ulcers, gastritis (an inflammation of the lining of the stomach), and stomach cancer, which have been linked to bacterial infection by helicobacter pylori.

Pancreatic Tissue.

Pancreatic organoids are organoids derived from pancreas. The pancreas is a gland organ that is both an endocrine gland (the “endocrine pancreas”), producing several important hormones, including insulin, glucagon, and somatostatin, as well as a digestive gland (the “exocrine pancreas”), secreting pancreatic juice containing digestive enzymes that assist the absorption of nutrients and the digestion in the small intestine. Endocrine function is mediated by the Islets of Langerhans, which appear by H&E staining as lightly stained, large, spherical clusters comprising alpha cells (15-20% of total islet cells; produce glucagon), beta cells (65-80% of total islet cells, produce insulin and amylin, and express pdx-1); delta cells (3-10% of total islet cells, produce somatostatin), PP cells (3-5% of total islet cells; produce pancreatic polypeptide), and epsilon cells (<1% of total islet cells; produce ghrelin). Exocrine function is mediated by the acini of the pancreas, which appear by H&E staining as darker stained, small, berry-like clusters. The acini comprise centroacinar cells, spindle-shaped cells that secrete an aqueous bicarbonate solution under stimulation by the hormone secretin. They also secrete mucin. Associated with the acini are tubes that deliver enzymes produced by the acinar cells into the duodenum; these tubes are lined with an epithelial lining of ductal cells, which express CK19 and CA19-9.

A number of diseases can be modeled using pancreatic organoids. These include, without limitation, pancreatic cancers, including those arising from the exocrine pancreas (pancreatic acinar cell carcinomas, or adenocarcinomas) and those arising from the islet cells (neuroendocrine tumors); diabetes, including type 1 diabetes in which there is direct damage to the endocrine pancreas that results in insufficient insulin synthesis and secretion, and type 2 diabetes mellitus, which is characterized by the ultimate failure of pancreatic β cells to match insulin production with insulin demand; and exocrine pancreatic insufficiency (the inability to properly digest food due to a lack of digestive enzymes made by the pancreas; occurs in cystic fibrosis and Shwachman-Diamond syndrome).

Bladder Tissue.

Bladder organoids are organoids derived from bladder tissue. The bladder is part of the urinary system, and consists of four structurally distinct tissue layers. The outermost of these, known as the serosal or tunica seros is derived from the peritoneum and covers only the upper and lateral surfaces of the bladder. Adjacent to and inward of the serosa layers is the muscle layer of the bladder, also known as the tunica muscularis or, more commonly, the “detrusor muscle” for its function in expelling urine from the bladder. Internal to the tunica muscularis is the submucosal layer, also known as the lamina propria. This layer consists of blood and lympathic vessels and nerves within a stroma of fibrous connective that join the tunica muscularis to the innermost of the bladder tissue layers, the tunica mucosa or mucosal layer. Internal to the submucosal layer is the mucosal layer, the innermost tissue of the bladder. The epithelial tissue layer of the bladder consists of from five to seven strata of transitional epithelial cells, also called urothelial cells. These cells appear cuboidal with a domed apex; when the bladder fills, they appear flat, irregular, and squamous. The uppermost cells of the urothelium at the inner surface of the bladder are known as umbrella cells. These cells, which extend over smaller cells in the new lower layer epithelium, are impermeable, resistant to infection and to the adherence of many foreign substances and thus provide protection for underlying cells of the urothelium. Additionally, umbrella cells secrete a protective substance known as mucin, which protects the underlying bladder cell from irritating substances present in urine. Urothelial cells are described in greater detail in Mauney J R et al. (2010) All-trans retinoic acid directs urothelial specification of murine embryonic stem cells via GATA4/6 signaling mechanisms. PLoS One 5(17):e11513. Terminal differentiation of bladder tissue is marked by the expression of uroplakin III, e-cadherin and CK8.

Bladder organoids find use in the study of a number of diseases, and the identification of therapies to treat them, including but not limited to bladder cancer, e.g. urothelial cell carcinoma, a type of cancer that typically occurs in the kidney, urinary bladder, and accessory organs; infection, e.g. cystitis cystica, a chronic cystitis glandularis accompanied by the formation of cysts; and interstitial cystitis, a bladder disease characterized by a bladder wall infiltrated by inflammatory cells resulting in ulcerated mucosa and scarring, spasm of the detrusor muscle, hematuria, urgency, increased frequency, and pain on urination.

Liver Tissue.

Liver organoids are organoids derived from liver tissue. The liver plays a major role in metabolism and has a number of functions in the body, including glycogen storage, decomposition of red blood cells, plasma protein synthesis, hormone production, and detoxification.

The liver comprises hepatocytes, which occupy almost 80% of the total liver volume, and nonparenchymal liver cells, which contribute only 6.5% to the liver volume, but 40% to the total number of liver cells. Hepatocytes are identifiable by their expression of Liver fatty-acid-binding protein (L-FABP), Cytochrome p450s and GSTp. The nonparenchymal cells, which are localized in the sinusoidal compartment of the tissue, include three different cell types: sinusoidal endothelial cells (SEC), Kupffer cells (KC), and hepatic stellate cells (HSC, formerly known as fat-storing cells, Ito cells, lipocytes, perisinusoidal cells, or vitamin A-rich cells). A self-renewing cell that resides in the liver and can give rise to these different cell types has been identified. In mouse, this cell is c-Met⁺CD49^{+/low} wc-Kit⁻CD45⁻TER119⁻; see, e.g., Suzuki, A., et al. (2002). Clonal identification and characterization of self-renewing pluripotent stem cells in the developing liver. J. Cell Biol. 156, 173-184.

Diseases and disorders affecting the liver that may be studied with organoids prepared by the subject methods and used in screens of the subject methods include infections, e.g. hepatitis infection; alcohol damage, fatty liver disease, cirrhosis, cancer, drug damage, and pediatric diseases, e.g. biliary atresia, alpha-1 antitrypsin deficiency, alagille syndrome, progressive familial intrahepatic cholestasis, and Langerhans cell histiocytosis.

Kidney Tissue.

Kidney organoids are organoids derived from kidney tissue. They are essential in the urinary system and also serve homeostatic functions such as the regulation of electrolytes, maintenance of acid-base balance, and regulation of blood pressure (via maintaining salt and water balance). They serve the body as a natural filter of the blood, and remove wastes which are diverted to the urinary bladder. In producing urine, the kidneys excrete wastes such as urea and ammonium; the kidneys also are responsible for the reabsorption of water, glucose, and amino acids. The kidneys also produce hormones including calcitriol, erythropoietin, and the enzyme renin.

A number of different types of cells exist in the kidney. They include, for example, the glomerulus parietal cell (squamous epithelial cells that line the outside of the Bowman's capsule); the glomerulus podocyte (cells of the Bowman's capsule that interface with the capillaries of the glomerulus; many coated vesicles and coated pits can be seen along the basolateral domain of the podocytes, indicating a high rate of vesicular traffic in these cells); the proximal tubule brush border cell (lines the luminal surface of the proximal tubule segment of the nephron, and has an apical surface of densely packed microvilli readily visible under the light microscope, which facilitates their resorptive function as well as flow-sensing within the lumen); the Loop of Henle thin segment cell; the thick ascending limb cell (expresses the sodium-potassium-2 chloride cotransporter (NKCC), which allows the kidney to produce concentrated urine when an individual has gone without water); the distal tubule cell (the target of thiazides that treat high blood pressure, it expresses the thiazide-sensitive sodium chloride cotransporter (TSC), and is responsible for reabsorbing about 5% of the sodium filtered by the kidney each day); the principal cell of the collecting duct (predominantly responsible for sodium reabsorption and potassium secretion in the kidney); the intercalated cells of the collecting duct (alpha intercalated cells, responsible for secreting excess acid and reabsorbing base in the form of bicarbonate; and beta intercalated cells, responsible for secreting excess base (bicarbonate) and reabsorbing acid); and the interstitial kidney cell. Terminal differentiation of kidney tissue is marked by the expression of Aquaporin 2 and Ksp-cadherin.

One diseases of interest affecting the kidney that may be studied with organoids prepared by the subject methods and used in screens of the subject methods is chronic kidney disease, diagnosed by a blood test for creatinine, which indicates a falling filtration rate and as a result, a decreased capability of the kidney to excrete waste products. Others include, without limitation, kidney cancer and kidney stones.

Tissue may be obtained by any convenient method, e.g. by biopsy, e.g. during endoscopy, during surgery, by needle, etc., and is typically obtained as aseptically as possible. Upon removal, tissue is immersed in ice-cold buffered solution, e.g. PBS, Ham's F12, MEM, culture medium, etc. Pieces of tissue are minced to a size less than about 1 mm³, and may be less than about 0.5 mm³, or less than about 0.1 mm³. The minced tissue is mixed with a gel substrate, e.g. a collagen gel solution, e.g. Cellmatrix type I-A collagen (Nitta Gelatin Inc.); a matrigel solution, etc. Subsequently, the tissue-containing gel substrate is layered over a layer of gel (a “foundation layer”) in a container with a lower semi-permeable support, e.g. a membrane, supporting the foundation gel layer, and the tissue-containing gel substrate is allowed to solidify. This container is placed into an outer container containing a suitable medium, for example HAMs F-12 medium supplemented with fetal calf serum (FCS) at a concentration of from about 1 to about 25%, usually from about 5 to about 20%, etc.

The arrangement described above allows nutrients to travel from the bottom, through the membrane and the foundation gel layer to the gel layer containing the tissue. The level of the medium is maintained such that the top part of the gel, i.e. the gel layer containing the explants, is not submerged in liquid but is exposed to air. Thus the tissue is grown in a gel with an air-liquid interface (FIG. 1A). A description of an example of an air-liquid interface culture system is provided in Ootani et al. in Nat. Med. 2009 June; 15(6):701-6, the disclosure of which is incorporated herein in its entirety by reference.

In some embodiments, tissue is grown in vitro from pluripotent stem cells, e.g. embryonic stem cells (ESCs), embryonic germ cells (EGCs), induced pluripotent stem cells (iPSCs). Any convenient method may be followed for the induction of the desired tissue from pluripotent stem cells; see, for example, Spence, J R et al. (2011) Nature 470(7332):105-9, for methods for growing intestinal cells from iPSCs; Wang, D. et al. (2007) Proc. Acad. Natl. Sci. USA 104(11):4449-4454 for methods for growing alveolar cells from iPSCs; or Mauney J R et al. (2010) All-trans retinoic acid directs urothelial specification of murine embryonic stem cells via GATA4/6 signaling mechanisms. PLoS One 5(17):e11513, for methods for growing bladder cells from iPSCs. Once the differentiation of pluripotent cells is observed, the engineered tissue is transferred to the gel substrate and treated as described above for culturing in the air-liquid interface culture system.

Explants cultured in this way may be sustained for over a year at physiological temperatures, e.g. 37° C., in a humidified atmosphere of, e.g. 5% CO₂ in air. Medium is changed about every 10 days or less, e.g. about 1, 2, or 3 days, sometimes 4, 5, or 6 days, in some instances 7, 8, 9, 10, 11 or 12 days, usually as convenient.

The continued growth of explants may be confirmed by any convenient method, e.g. phase contrast microscopy, stereomicroscopy, histology, immunohistochemistry, electron microscopy, etc. In some instances, cellular ultrastructure and multi-lineage differentiation may be assessed. Ultrastructure of the intestinal explants in culture can be determined by performing Hematoxylin-eosin staining, PCNA staining, electron microscopy, and the like using methods known in the art. Multi-lineage differentiation can be determined by performing labeling with antibodies to terminal differentiation markers, e.g. as described in greater detail below. Antibodies to detect differentiation markers are commercially available from a number of sources.

In some embodiments, the growth of the explants in culture, e.g. pancreatic organoids, liver organoids, bladd organoids, lung organoids, etc., may be stimulated by introducing R-spondin into the culture medium. R-spondin1 (Rspo1, Genbank Accession NP_{—}001033722) is a secreted glycoprotein which synergizes with Wnt to activate β-catenin dependent signaling (Kim et al., 2005, Kim et al., 2006). Explants cultured by the subject methods that are exposed to RSpo1 exhibit increased growth. The factors may be added to the culture at a concentration of at least about 500 ng/ml, at least about 0.5 μg/ml, at least about 50 μg/ml and not more than about 1 mg/ml, with change of medium every 1-2 days.

In some embodiments, the cells in the cultured explants may be experimentally modified. For example, the explant cells may be modified by exposure to viral or bacterial pathogens, e.g. to develop a reagent for experiments to assess the anti-viral or anti-bacterial effects of therapeutic agents. The explant cells may be modified by altering patterns of gene expression, e.g. by providing reprogramming factors to induce pluripotency or otherwise alter differentiation potential, or to determine the effect of a gain or loss of gene activity on the ability of cells to form an explant culture or on the ability of cells to undergo tumor transformation. The explant cells may be modified such that they are transformed into proto-oncogenic or oncogenic cells, e.g. by providing cancer drivers—oncogenic factors or inhibitors of tumor suppressor genes, e.g. nucleic acids for the overexpression of Kras^{G12D}; nucleic acids that suppress expression of APC, p53, or Smad4, etc—for example, to assess the effects of therapeutic agents on tumors.

Experimental modifications may be made by any method known in the art, for example, as described below with regard to methods for providing candidate agents that are nucleic acids, polypeptides, small molecules, viruses, etc. to explants and the cells thereof for screening purposes.

### Utility

Organoids prepared by the subject methods find use in many applications. For example, cancer, ischemia, congenital syndromes, trauma, and inflammation can produce functional loss or mandate physical resection of large sections of patient tissue extensive enough to compromise organ physiology. The ability to grow explants of mammalian tissue in vitro to be placed back into such patients or to be used as a source of tissue-specific stem cells for transplantation into such patients is a valuable treatment option. Such cells can augment the ex vivo expansion of tissue, providing an autologous source of engineered tissue and/or tissue stem cells. As another example, organoids prepared by the subject methods may be used to predict the responsiveness of an individual, e.g. an individual with cancer, with an infection, etc., to a therapy. As another example, organoids prepared by the subject methods may be used in basic research, e.g. to better understand the basis of disease, and in drug discovery, e.g. as reagents in screens such as those described further below. Organoids are also useful for assessing the pharmacokinetics and pharmacodynamics of an agent, e.g. the ability of a mammalian tissue to absorb an active agent, the cytotoxicity of agents on primary mammalian tissue or on oncogenic mammalian tissue, etc.

### Screening Methods

In some aspects of the invention, methods and culture systems are provided for screening candidate agents or cells for an activity of interest. In these methods, candidate agents or cells are screened for their effect on cells in the organoids of the invention. Organoids of interest include those comprising unmodified cells, and those comprising experimentally modified cells as described herein, including cancer cells, infected cells, cells treated with potentially cytotoxic agents and the like. Also included are stem cells, cancer stem cells, progenitor cells or differentiated or oncogenically transformed progeny thereof.

The effect of an agent or cells is determined by adding the agent or cells to the cells of the cultured explants as described herein, usually in conjunction with a control culture of cells lacking the agent or cells. The effect of the candidate agent or cell is then assessed by monitoring one or more output parameters. Parameters are quantifiable components of explants or the cells thereof, particularly components that can be accurately measured, in some instances in a high throughput system. For example, a parameter of the explant may be the growth, differentiation, gene expression, proteome, phenotype with respect to markers etc. of the explant or the cells thereof, e.g. any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

In some embodiments, candidate agent or cells are added to the cells within the intact organoid. In other embodiments, the organoids are dissociated, and candidate agent or cells is added to the dissociated cells. The cells may be freshly isolated, cultured, genetically altered as described above; or the like. The cells may be environmentally induced variants of clonal cultures: e.g. split into independent cultures and grown into organoids under distinct conditions, for example with or without pathogen; in the presence or absence of other cytokines or combinations thereof. The manner in which cells respond to an agent, particularly a pharmacologic agent, including the timing of responses, is an important reflection of the physiologic state of the cell.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Candidate agents of interest for screening also include nucleic acids, for example, nucleic acids that encode siRNA, shRNA, antisense molecules, or miRNA, or nucleic acids that encode polypeptides. Many vectors useful for transferring nucleic acids into target cells are available. The vectors may be maintained episomally, e.g. as plasmids, minicircle DNAs, virus-derived vectors such cytomegalovirus, adenovirus, etc., or they may be integrated into the target cell genome, through homologous recombination or random integration, e.g. retrovirus derived vectors such as MMLV, HIV-1, ALV, etc. Vectors may be provided directly to the subject cells. In other words, the pluripotent cells are contacted with vectors comprising the nucleic acid of interest such that the vectors are taken up by the cells.

Methods for contacting cells with nucleic acid vectors, such as electroporation, calcium chloride transfection, and lipofection, are well known in the art. Alternatively, the nucleic acid of interest may be provided to the subject cells via a virus. In other words, the pluripotent cells are contacted with viral particles comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PAl2 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject CD33+ differentiated somatic cells are targeted by the packaged viral particles. Methods of introducing the retroviral vectors comprising the nucleic acid encoding the reprogramming factors into packaging cell lines and of collecting the viral particles that are generated by the packaging lines are well known in the art.

Vectors used for providing nucleic acid of interest to the subject cells will typically comprise suitable promoters for driving the expression, that is, transcriptional activation, of the nucleic acid of interest. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing reprogramming factors to the subject cells may include genes that must later be removed, e.g. using a recombinase system such as Cre/Lox, or the cells that express them destroyed, e.g. by including genes that allow selective toxicity such as herpesvirus TK, bcl-xs, etc

Candidate agents of interest for screening also include polypeptides. Such polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

If the candidate polypeptide agent is being assayed for its ability to inhibit aggregation signaling intracellularly, the polypeptide may comprise the polypeptide sequences of interest fused to a polypeptide permeant domain. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

If the candidate polypeptide agent is being assayed for its ability to inhibit aggregation signaling extracellularly, the polypeptide may be formulated for improved stability. For example, the peptides may be PEGylated, where the polyethyleneoxy group provides for enhanced lifetime in the blood stream. The polypeptide may be fused to another polypeptide to provide for added functionality, e.g. to increase the in vivo stability. Generally such fusion partners are a stable plasma protein, which may, for example, extend the in vivo plasma half-life of the polypeptide when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the polypeptide also will be produced and employed as a multimer having substantially the same structure as the stable plasma protein precursor. These multimers will be homogeneous with respect to the polypeptide agent they comprise, or they may contain more than one polypeptide agent.

The candidate polypeptide agent may be produced from eukaryotic produced by prokaryotic cells, it may be further processed by unfolding, e.g. heat denaturation, DTT reduction, etc. and may be further refolded, using methods known in the art. Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine. The polypeptides may have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The candidate polypeptide agent may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Alternatively, the candidate polypeptide agent may be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In some cases, the candidate polypeptide agents to be screened are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured.

Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one and usually a plurality of explant or cell samples, usually in conjunction with explants not contacted with the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow-through method. Alternatively, the agents can be injected into the explant, e.g. into the lumen of the explant, and their effect compared to injection of controls.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the growth rate.

Screens for Agents with Anti-Viral or Anti-Bacterial Activity.

The subject organoids are useful for screening candidate agents for anti-viral or anti-bacterial activity. In screening assays for assessing a candidate agent's ability to inhibit, or “neutralize”, a biologically active pathogen, the subject organoids are contacted with the agent of interest, for example in the presence of a pathogen (bacterial, viral, fungal), and the effect of the agent assessed by monitoring one or more output parameters, e.g. cell survival, explant growth, explant ultrastructure, viral titer, bacterial growth, toxicology testing, immunoassays for protein binding, differentiation and functional activity, production of hormones; and the like. The cells may be freshly isolated, cultured, genetically altered as described above; or the like. The cells may be environmentally induced variants of clonal cultures: e.g. split into independent cultures and grown under distinct conditions, for example with or without pathogen; in the presence or absence of other cytokines or combinations thereof. The manner in which cells respond to an agent, particularly a pharmacologic agent, including the timing of responses, is an important reflection of the physiologic state of the cell.

Screens for Agents with Anti-Tumorigenic or Anti-Tumoral Activity.

In some embodiments, a candidate agent is screened for activity that is anti-tumorigenic (i.e. inhibiting cancer initiation) or anti-tumoral (i.e. inhibiting cancer progression, e.g. proliferation, invasion, metastasis). In such embodiments, the explant culture includes cancer cells, including cells suspected of being cancer stem cells. Assessment of anti-tumor activity may include measurements of one or more parameters including explant growth, the rate or extent of cell proliferation, the rate or extent of cell death, etc.

In some embodiments, the cancer cells are provided to the organoid, i.e. the organoid is contacted with the cancer cell, e.g. a cancer stem cell, and the candidate agent's anti-tumorigenic activity is assessed on that cancer cell in the context of the organoid. Methods for purifying cancer stem cells have been previously described, for example in US20070292389A1 and US2070238127A1. US20070292389A1 describes purification of cancer stem cells from solid epithelial tumors. The method of purification and amplification of cancer stem cells disclosed in US20070292389A1 is herein incorporated by reference.

In some embodiments, the cancer cells, e.g. cancer stem cells, are naturally occurring. In other words, the cancer cells spontaneously formed in the tissue, e.g. before the tissue was obtained from the mammal, or during explant culturing.

In some embodiments, non-transformed cells of the explant are experimentally modified prior to, or during the explant culture period by altering patterns of gene expression by introducing cancer drivers (e.g. expressible coding sequences, anti-sense and RNAi agents, etc.) that provide for transformation of the explant cells into carcinomas, e.g. APC; Kras; p53; SMAD4; etc. The experimentally modified cells are useful for investigation of the effects of therapeutic agents for tumor therapy and identification of new therapeutic molecular targets. Such methods allow investigation of cancer initiation and treatment. Candidate agents of interest include, without limitation, chemotherapy, monoclonal antibodies or other protein-based agents, radiation/radiation sensitizers, cDNA, siRNA, shRNA, small molecules, and the like.

Screens for Agents to Prevent or Treat Disease.

Other examples of screening methods of interest include methods of screening a candidate agent for an activity in treating or preventing a disease. In such embodiments, the explant models the disease, e.g. the explant may have been obtained from a diseased tissue. For example, the explant may be obtained from an individual having a disease to determine if that agent will prevent or treat disease in that individual. In other words, the screen is used to predict the responsiveness of an individual to a known therapy, e.g. an anti-viral therapy, an anti-bacterial therapy, a cancer therapy (e.g. an anti-tumorigenic or anti-tumoral therapy), etc. In such instances, a sample, e.g. a human tumor sample, may be taken from an individual; the sample may be cultured using the subject methods; the cultured sample may be contacted with the therapeutic agent, e.g. chemotherapy, antibody therapeutic, small molecule therapeutic; and the effect of the therapeutic agent on the sample may be determined by measuring one or more parameters, where an effect of the therapeutic agent on the sample is predictive of the effect that the therapeutic agent will have on the individual. As another example, the explant may be a tissue from a healthy individual that is experimentally modified to model the disease by, e.g., genetic mutation, e.g. to determine the responsiveness of an individual to therapy should that individual develop a disease. Parameters such as explant growth, cell proliferation, cell viability, cell ultrastructure, tissue ultrastructure, etc. find particular use as output parameters in such screens.

Screens to Determine the Pharmacokinetics and Pharmacodynamics of Agents.

Other examples include methods of screening a candidate agent for toxicity to tissue. In these applications, the cultured explant is exposed to the candidate agent or the vehicle and its growth and viability is assessed. In these applications, analysis of the ultrastructure of the explants is also useful.

Screens for Cells with Stem Cell Activity.

The identification of cells that are stem cells or that possess the potential to become stem cells that will differentiate into the cell types of a tissue of interest is valuable for tissue repair and tissue augmentation, e.g. after injury, disease, transection, etc. Candidate cells are screened by adding the cells to the organoids described herein, usually in conjunction with a control organoid culture lacking the candidate cell. Increases in growth, proliferation, and/or multi-lineage differentiation above basal levels in explants contacted with candidate cells as compared to explants not contacted with candidate cells is indicative that the candidate cell is a stem cell or has the potential to develop into a stem cells.

Candidate cells can be detectably marked, for example via expression of a marker such as GFP or β-galactosidase. Candidate cells marked via expression of GFP are derived by standard techniques. GFP transduced candidate cells can be generated by techniques well known in the art, for example using a viral vector expressing GFP. Labeled candidate cells may be co-cultured with non-labeled explants. The candidate cells may be mixed with the explant culture prior to mixing with gel (and subsequent long term culture). Alternatively the candidate cells may be mixed with explants that have been grown in vitro for some length of time, in which case they may be injected into the explant, e.g. a lumen of the explant. Cells may be introduced in a limiting dilution, or as a population, e.g. 1, 5, 10, 100, 500, 1000 or more cells per culture. The co-culture of candidate cells and explant may be culture for at least about 1, 2, 3, 4, 5, 6, 7, 8, 9, 10, 11, 12, or more weeks prior to evaluation for differentiation into epithelial cell lineages.

The assessment of the candidate cells may be performed by visual observation, e.g. the stimulation of growth of explants in culture compared to the explants not co-cultured with the candidate cells. Alternatively, expression of various differentiation markers can be evaluated. Immunofluorescence can be performed using antibodies against differentiation markers specific for the tissue under study. Dual color immunofluorescence may be performed with the intrinsic GFP signal to confirm co-localization of differentiation markers with candidate cells. Another criteria for stem cell function is self-renewal, with concomitant long-term proliferation and reconstitution activities. Long-term proliferation of GFP-transduced candidate cells within the explants can be assayed both in vitro and in vivo, and compared to control explants without the candidate cells.

Methods of in vitro analysis include, without limitation, serial passage of explant:candidate co-cultures. For example, organoids may be transplanted intact or subdivided as fragments into fresh gel followed by continued culture. Explants thus transplanted may eventually be harvested and sectioned for microscopic or visual analysis. Serial transplantability of explants co-cultured with candidate cells are compared to that of explants grown without candidate cells.

Methods of in vivo analysis include various methods where explants are transferred to an in vivo environment. In some embodiments, organoids are generated using the methods described above, extracted from the gel, and implanted into the organ or subcutaneously into an experimental animal, e.g. syngeneic or immunodeficient mice, then allowed to grow for a suitable period of time, e.g. at least about 1 week, at least about 2 weeks, at least about 3-4 weeks, at least about 1, 2, 3, 4 or more months, etc. This assay can be modified to utilize various marker systems, e.g. luciferase expressing cells that permit periodic non-invasive imaging after luciferin injection. Growth and serial transplantability is compared between explants with and without candidate cells.

### High Throughput Screens

In some aspects of the invention, methods and culture systems are provided for screening candidate agents in a high-throughput format. By “high-throughput” or “HT”, it is meant the screening of large numbers of candidate agents or candidate cells simultaneously for an activity of interest. By large numbers, it is meant screening 20 more or candidates at a time, e.g. 40 or more candidates, e.g. 100 or more candidates, 200 or more candidates, 500 or more candidates, or 1000 candidates or more.

In some embodiments, the high throughput screen will be formatted based upon the numbers of wells of the tissue culture plates used, e.g. a 24-well format, in which 24 candidate agents (or less, plus controls) are assayed; a 48-well format, in which 48 candidate agents (or less, plus controls) are assayed; a 96-well format, in which 96 candidate agents (or less, plus controls) are assayed; a 384-well format, in which 384 candidate agents (or less, plus controls) are assayed; a 1536-well format, in which 1536 candidate agents (or less, plus controls) are assayed; or a 3456-well format, in which 3456 candidate agents (or less, plus controls) are assayed. High throughput screens formatted in this way may be achieved by using, for example, transwell inserts. Transwell inserts are wells with permeable supports, e.g. microporous membranes, that are designed to fit inside the wells of a multi-well tissue culture dish. In some instances, the transwells are used individual. In some instances, the transwells are mounted in special holders to allow for automation and ease of handling of multiple transwells at one time.

To achieve the numbers of organoids necessary to perform a high-throughput screen, a primary organoid (that is, an organoid that has been cultured directly from tissue fragments) is dissociated into a single cell suspension and replated across multiple transwells to generate secondary organoids in a multiwell format. Dissociation may be by any convenient method, e.g. manual treatment (trituration), or chemical or enzymatic treatment with, e.g. EDTA, trypsin, papain, etc. that promotes dissociation of cells in tissue. The dissociated organoid cells are then replated in transwells at a density of 10,000 or more cells per 96-well transwell, e.g. 20,000 cells or more, 30,000 cells or more, 40,000 cells or more, or 50,000 cells or more. Additional iterations of dissociation and plating may be performed to achieve the desired numbers samples of organoids to be treated with agent.

In some embodiments, the secondary (or tertiary, etc.) organoids may be cultured first, after which candidate agents or cells are added to the organoid cultures and parameters reflective if a desired activity are assessed. In other embodiments, the candidate agents or cells are added to the dissociated cells at replating. This latter paradigm may be particularly useful for example for assessing candidate agents/cells for an activity that impacts the differentiation of cells of the developing organoid. Any one or more of these steps may be automated as convenient, e.g. robotic liquid handling for the plating of explants, addition of medium, and/or addition of candidate agents; robotic detection of parameters and data acquisition; etc.

The following examples are put forth so as to provide those of ordinary skill in the art with a complete disclosure and description of how to make and use the subject invention, and are not intended to limit the scope of what is regarded as the invention.

### EXPERIMENTAL

Cancer arises from the acquisition and concerted action of multiple mutations and genomic aberrations in discrete combinations of tumor suppressors and oncogenes (e.g. “drivers”). These synergistic combinations of specific drivers (e.g. networks) lead to tumorigenesis and define the individual biological properties of any given cancer. In the post-genomic era, a major challenge will be (1) biologically delineating the specific drivers combinations, that we refer to as “cancer driver modules”, that are responsible for the underlying biology of any given cancer and (2) determining how these modules can be exploited therapeutically. This will involve assessing the combined biological effects of specific combinations of well-defined and putative drivers for any given type of cancer.

For any given individual malignancy, the combined, synergistic effect of these “networked” drivers is responsible for (1) neoplastic development and (2) determining the underlying cancer biology that mediates response to specific therapies. Cancer genomes show a remarkable degree of genomic variability even among the same histopathologic classification. The Cancer Genome Atlas Project (TCGA) has directly addressed this issue by using multiple genomic platforms to analyze hundreds of samples among the twenty tumor types and this population-based approach enables discrimination of potential drivers from passengers. However, even with the statistical power provided by interrogating large sample sets, post-analysis there are many candidate cancer drivers that must be experimentally assessed for their contributing role in cancer biology and their exploitation in therapy development. Ultimately, translation of the TCGA genetic and genomic findings for clinical application and therapeutic discovery will require the development of a robust cancer model system that can be specifically engineered with candidate cancer drivers. Only then can one assess the biological properties of these drivers and determination of whether sets of specific cancer drivers offer an opportunity for novel therapeutic intervention.

Historically, the in vitro validation of novel oncogenes and tumor suppressors has utilized 2D culture of transformed cell lines. However, in vitro validation of putative cancer drivers from TCGA and similar genome-scale surveys would ideally utilize primary cells as opposed to cell lines, which have long-term passage and genetic heterogeneity, growth in 2D monolayer and lack of modeling of the tumor microenvironment (Ashworth, A., et al. (2001) Genetic interactions in cancer progression and treatment. Cell 145, 30-38; Haber, D. A., et al. (2011) The evolving war on cancer. Cell 145, 19-24). A similar reliance on transformed cell lines has also existed for drug screening, i.e. “NIH60” (Chabner, B. A. & Roberts, T. G., Jr. 2005. Timeline: Chemotherapy and the war on cancer. Nat Rev Cancer 5, 65-72). Primary culture models have been vastly underutilized for both functional validation of oncogenic loci and for therapeutic screening. This has been due in no small part to a lack of appropriately robust and scalable culture methods for numerous organ systems, which has rendered it impossible to initiate carcinogenesis in vitro from many primary tissues, thus precluding functional oncogene validation and therapeutic screening applications. What is needed is a single method for culturing primary organoids that (1) exhibit long-term proliferation and multi-lineage differentiation, and (2) can be transformed with complex oncogenic driver modules, which can be applied across diverse tissues without modification.

Described herein is a single primary 3D “organoid” culture method using air-liquid interfaces (ALI), which is broadly applicable to numerous explanted tissues with long-term proliferation and multi-lineage differentiation. Notably, this single methodology allows diverse primary organoids to be transformed with up to 4 simultaneous oncogenic events by combined genetic and viral strategies, and is further scalable to high-throughput (HT) format for TCGA gene validation and drug discovery applications.

The following examples demonstrate the applicability of the method to various mammalian tissues and a number of cancers. The method utilizes a single 3D air-liquid interface method that (1) accurately recapitulates normal tissue architecture and differentiation in organoids and (2) allows robust engineering of multiple transforming oncogenic events in a variety of tissues. We have used the method to generate organotypic cultures for colon, lung, stomach, pancreas and bladder.

In addition, the following examples demonstrate that, using the identical method without modification, these cultures may be used to establish in vitro models for colon, lung and gastric adenocarcinoma. These organoid models of diverse cancer are a significant advance over conventional transformed cell lines as they are (a) primary tissue, (b) have not been repetitively passaged, (c) are grown in a more physiologic 3D environment, (d) accurately recapitulate multilineage differentiation and cellular ultrastructure of the cognate normal organs, and (e) allow multiple concomitant oncogene manipulation. Our ability to engineer complex combinatorial driver modules within physiologic 3D primary organoid culture, via (1) Cre-mediated activation of floxed alleles and/or (2) robust retroviral co-infection, is a major innovation that strongly addresses the need to model the concerted action of oncogenic loci in TCGA data sets, whose massive complexity is compounded by combinatorial driver action.

The following examples also demonstrate the adaptation of these oncogene-engineered primary 3D organoids across diverse tissues in a multiwell format allowing viral transduction, compound screening and measurement of proliferation. These innovations greatly facilitate high-throughput approaches to combinatorial gene validation and to drug screening/responses including chemotherapy and targeted biologics.

Finally, the following examples also demonstrate methods for human organoid generation, using hESC-derived tissue and ecotropic retrovirus for gene delivery. This safety issue is especially significant when contemplating large-scale/high-throughput validation of unknown genes.

### Example 1

### General Methodology for the Preparation of Air-Liquid Interface Cultures

Tissue is procured under sterile conditions, minced and mixed with type I collagen gel. Subsequently, these explant containing gels are poured onto transwell cell culture inserts with a collagen gel layer. Transwell cell culture inserts are available commercially from a number if resources e.g. Corning, Signaaldrich. These cell culture inserts are placed into secondary outer dishes containing medium such as HAMs F-12 with 20% FCS. Medium is changed every 7 days. Organoids prepared in this manner may be maintained for a year or more.

Detailed Protocol for Explant Culture.

This culture system maintains the cultured cells embedded in the collagen gel under an air-liquid interface environment. Before preparing the tissue, an inner dish with collagen gel bottom layer should be made. The following procedure is done using Cellmatrix type I-A (Nitta Gelatin Inc.) as a premixed type I collagen gel, however, other products are able to use as an extracellular matrix, such as matrigel. The inner dish should have permeable and/or pored membrane bottom, such as a cell culture insert. We typically use Millicell culture plate inserts (Millicell-CM, Millipore Co.) or Falcon cell culture inserts (BD Co.) as the inner dish. All the following material scale/volume are variable and should be selected in accordance with the intended use. For example, a 1 ml of collagen gel solution is poured into a 30-mm diameter inner dish in combination with 60 mm diameter outer dish and 2 ml of culture media. If 10-mm diameter inner dish is applied, 0.3 ml of collagen gel solution is poured into the inner dish in combination with a 24-well outer dish and 0.5 ml of culture media. The inner dish is ready to use after the gel solidifies (see below).

Mammalian tissue, e.g. tissue from mice or humans, is removed with aseptic procedure. The removed tissue (typically 1 cm) is immediately immersed in ice-cold PBS or other culture media/tissue preservative solution such as Ham's F12 medium without serum. Tissues comprising a lumen are opened lengthwise and washed in ice-cold PBS (or other solution mentioned above) to remove all luminal contents.

The washed tissue is minced by iris scissors etc. on ice-cold plate such as a tissue culture plate lid. The final minced tissue has heterogenous size, but under 0.1 mm³ is suitable for culture. The tissue should be minced extensively so as to have an almost viscous appearance. This procedure should be done within 5 minutes to avoid cell damage and drying the tissue. The minced tissue is mixed in ice-cold, pre-solidified collagen gel solution.

The cell-containing collagen gel is poured onto the inner dish prepared as above. The inner dish is placed in the outer dish. The gel easily solidifies at 37° C. within 30 minutes. After solidifying the cell-containing gel, the culture media is poured into the outer dish. For a 1 ml of collagen gel solution poured into a 30-mm diameter inner dish, ≦2 ml of culture media should be added into the 60 mm diameter outer dish. At this point, the cultured cells should not be immersed in culture media. The cellular gel layer should exist above the medium level to create the air-liquid interface microenvironment.

Variable solution and antibiotics can be used for culture media. Ham's F12 is used herein, supplemented with 20% fetal calf serum and 50 μg/ml gentamicin. Variable substances such as protein or drug can be added in the culture media. The culture assembly is carried out over 30 to >350 days at 37° C. in a humidified atmosphere of 5% CO₂ in air. Medium is changed every 7 days, but the frequency may depend on cell numbers and if labile test growth factors are being added. Living culture cells can be observed by phase-contrast microscopy or stereo microscopy.

For histological analysis, the culture assembly can be fixed with variable solutions such as 4% PFA and embedded in paraffin. Deparaffinized cross sections can be stained with variable staining methods such as hematoxylin and eosin. Deparaffinized sections are able to be use for immunohistochemistry for variable antibodies. For ultrastructural analysis by transmission electron microscopy, the culture assembly can be fixed with 2.5% glutaraldehyde and 1% osmic acid, dehydrated with alcohol, and embedded in epoxy resin.

### Example 2

### Culturing Intestinal Organoids, and Introduction of Transforming Events into Organoids

Multiple simultaneous oncogenic events may be introduced into organoids from a variety of tissues by either: (1) Cre-mediated activation of floxed alleles in organoids from compound allele mice, and/or (2) retroviral gene transfer.

An example of Cre-mediated activation of floxed alleles in organoids was performed with intestinal organoids from APC^{flox/flox}; LSL KRas^{G12D}; p53^{flox/flox} mice. Neonatal colon explants cultured under air-liquid interface (ALI) prepared as described above resulted in expansive growth as epithelial spheres, with the apical side facing a central lumen, and sustained intestinal proliferation and multi-lineage differentiation over a range of 30 to >350 d. Further, the organoids exhibited spontaneous peristalsis, recapitulated the endogenous Wnt and Notch signaling of the intestinal stem cell (ISC) niche, and contained both Lgr5+(FIG. 1H) and Bmi1+ISC populations, which can generate all intestinal lineages in vivo (Sangiorgi, E. & Capecchi, M. R. (2008) Bmi1 is expressed in vivo in intestinal stem cells. Nat Genet. 40, 915-920; Barker, N., et al. (2007) Identification of stem cells in small intestine and colon by marker gene Lgr5. Nature 449(7165), 1003-1007; Barker, N., et al. (2008). The intestinal stem cell. Genes Dev 22, 1856-1864; Scoville, D. H., et al. (2008) Current view: intestinal stem cells and signaling. Gastroenterology 134, 849-864).

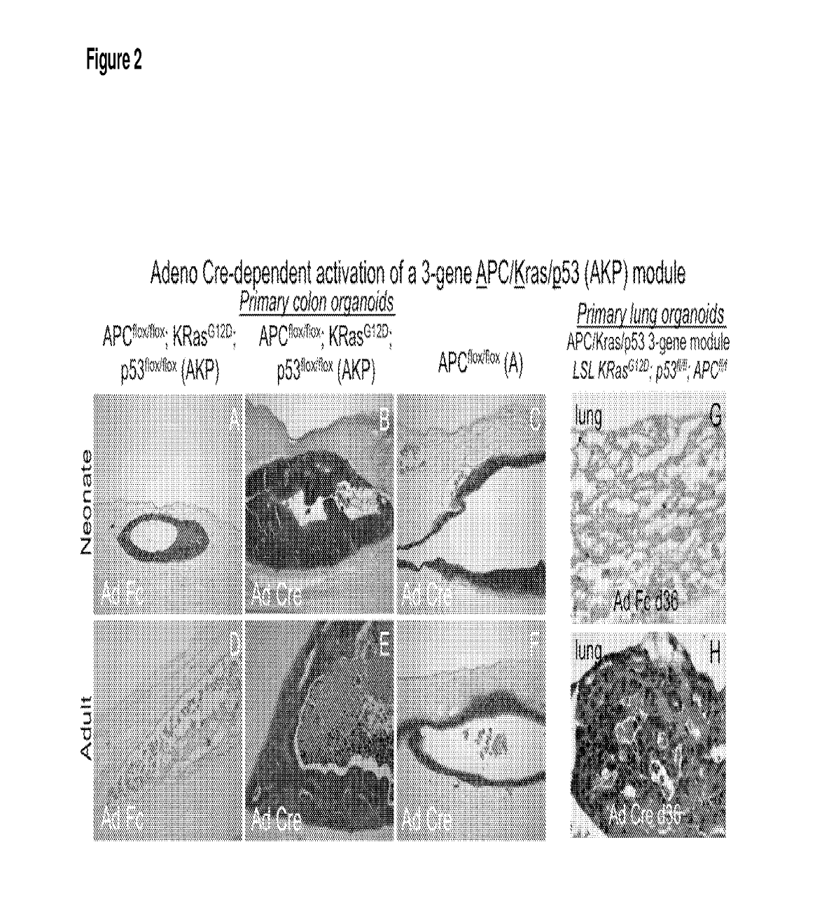

Adenovirus-Cre infection of neonatal colon organoids from APC^{flox/flox}; LSL KRas^{G12D}; p53^{flox/flox} mice resulted in in vitro deletion/activation of the APC/KRas^{G12D}/p53 3-oncogene module (“AKP”). This was accompanied by marked dysplasia (FIG. 2B) which was not seen with a control adeno Ad Fc encoding an antibody Fc fragment (FIG. 2A). Adult organoids grow extremely poorly in our system, but Ad-Cre but not Ad-Fc induced pronounced dysplasia in both adult APC^{flox/flox}; LSL KRas^{G12D}; p53^{flox/flox} colon and lung organoids, robustly rescuing the adult growth deficits (APC is mutated in lung adenoCa (Ding, L., et al. (2008). Somatic mutations affect key pathways in lung adenocarcinoma. Nature 455, 1069-1075; Greulich, H. (2010) The genomics of lung adenocarcinoma: opportunities for targeted therapies. Genes Cancer 1, 1200-1210). In both adult and neonatal colon organoids, the 3-gene AKP module was much more dysplastic than the 1-gene APC module (“A”) from Ad Cre treatment of APC^{fl/fl} organoids (FIG. 2B vs 2C; 2E vs 2F).

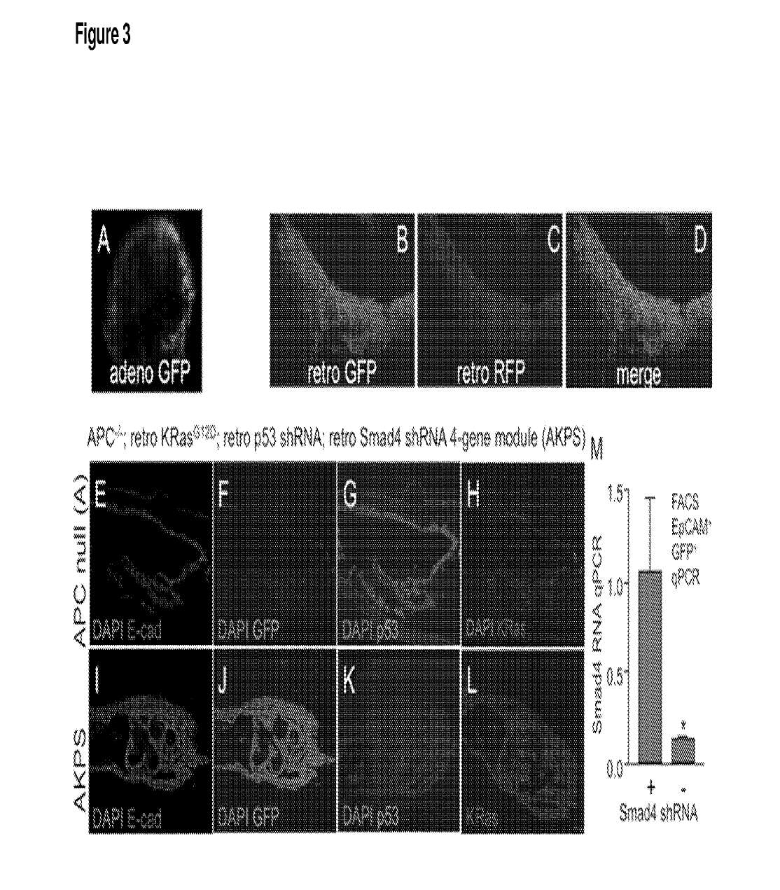

As an example of retroviral gene transfer, quantitative epithelial co-infection of colon organoids was demonstrated by co-infection with retrovirus GFP+retrovirus RFP (FIG. 3B-D). In colon, baseline APC loss-of-function mutations and subsequent mutations in KRAS/TP53 synergize to induce adenocarcinoma (Sansom, O. J., et al. (2006) Loss of Apc allows phenotypic manifestation of the transforming properties of an endogenous K-ras oncogene in vivo. Proc Natl Acad Sci USA 103, 14122-14127; Haigis, K. M., et al. (2008) Differential effects of oncogenic K-Ras and N-Ras on proliferation, differentiation and tumor progression in the colon. Nat Genet. 40, 600-608), comprising CRC multigenic modules. As an example, we engineered a 4-gene APC/KRas/p53/Smad4 (AKPS) module by infecting APC-null colon organoids (APC^{flow/flox}; villin-CreER+tamoxifen) with 3 ecotropic retroviruses encoding (1) KRas^{G12D} as a positive control dominantly-acting oncogene, (2) p53 shRNA and (3) Smad4 shRNA as positive-control tumor suppressors. p53 and Smad4 knockdown and KRas overexpression was confirmed (FIG. 3G-M) and the p53 and Smad4 shRNA viruses expressed their IRES GFP cassette (FIG. 3J).

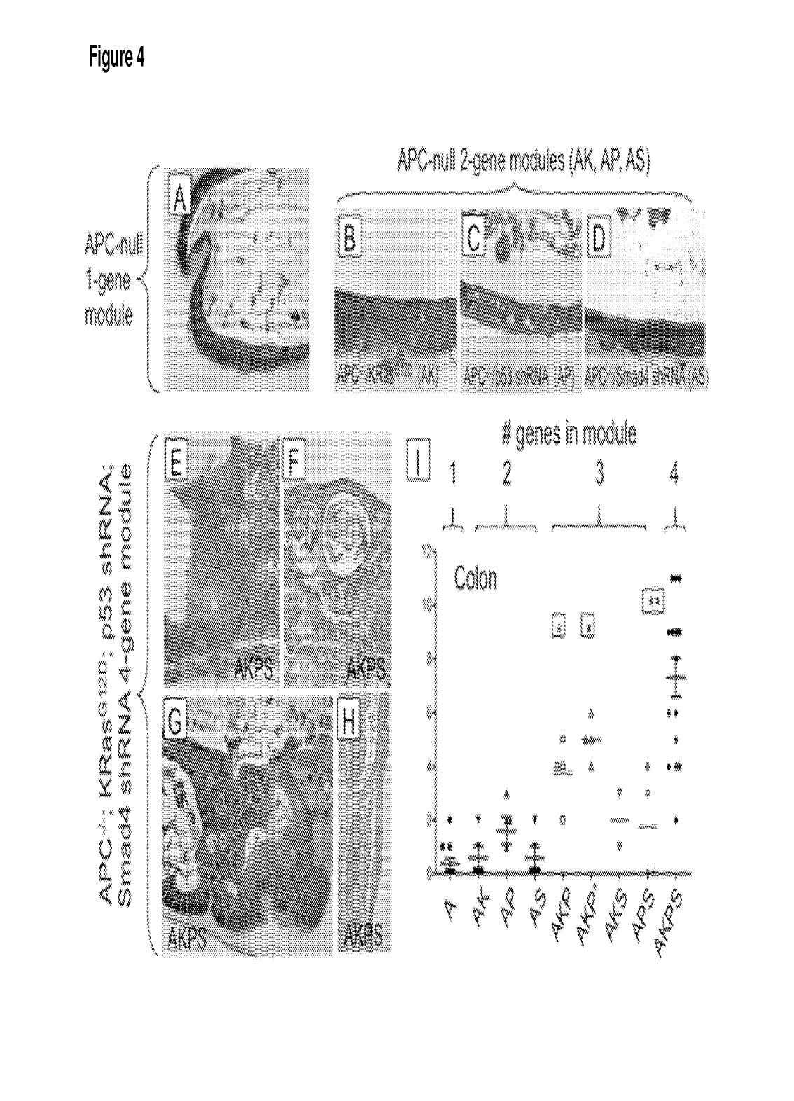

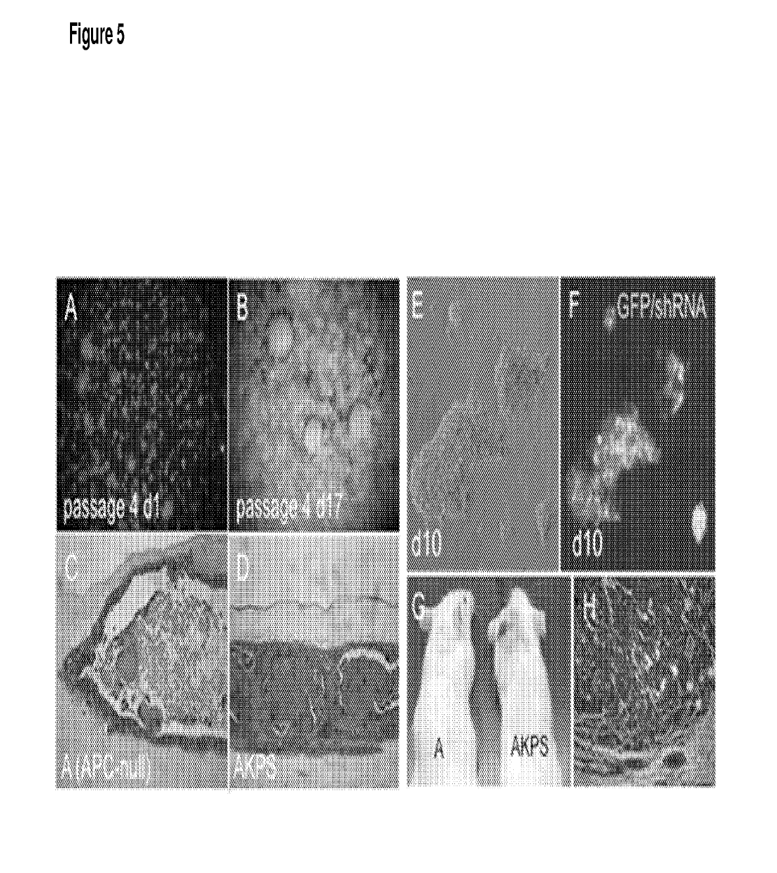

Organoids were used to explore oncogenicity of cancer driver modules of varying complexity. An objective dysplasia index with blinded evaluation incorporating proliferation, nuclear atypia, invasion and cellular stratification was developed. In primary colon organoids, 2-gene modules such as APC/KRas^{G12D} (AK), APC/p53 shRNA (AP) and APC/Smad4 shRNA (AS) elicited only minimal increases in dysplasia index versus APC deletion alone (A) (FIG. 4A-D, I), However, the retroviral 3-gene module AKP (APC/KRas/p53), induced increased dysplasia which was phenocopied by Ad Cre infection of APC^{flox/flox}; LSL KRas^{G12D}; p53^{flox/flox}; mice (AKP*) (c.f. FIG. 2B,E). Impressively, the AKPS four-gene module markedly transformed primary colon organoids with nuclear atypia, invasion, ranging from confluent sheets of cells to cribriform growth patterns with luminal necrosis and jagged infiltrating growth patterns which characterize human colon cancer (FIG. 4E-H) with dysplasia index vastly exceeding either 1- or 2- or 3-gene driver modules (P=0.007) (FIG. 4I). Focus formation and in vivo tumorigenicity were also assessed as parameters reflective of transformation. The AKPS organoids can be robustly passaged in ALI (>10 passages) (FIG. 5A, B) as confluent masses (FIG. 5D) vs. the spheroid “cystic” morphology from primary plating (FIG. 5C). In contrast, APC-null 1-gene organoids can only be passaged 2-3 times. AKPS organoids also exhibit focus formation on plastic and GFP-positivity from the retroviral IRES GFP cassettes (FIG. 5E, F). Further, AKPS organoids serially expanded in ALI can be transplanted subcutaneously into immunodeficient NSG mice (the APCflox/flox; villin-CreER mice are mixed background) (FIG. 5G, H); with robust tumor take with AKPS (8/8 mice) but not APC-null cells (0/8), indicating full oncogenic transformation.

These data represent the first successful transformation of primary colon organoids in vitro and support classical human CRC models where multiple transforming events are required for development of invasive carcinoma (Fearon, E. R. & Vogelstein, B. (1990) A genetic model for colorectal tumorigenesis. Cell 61, 759-767) and validate crucial positive controls for our primary colon organoids in screening TOGA driver modules. Such in vitro methods for assaying driver loci in primary intestinal culture have not previously existed.

### Example 3

### Gastric Cultures

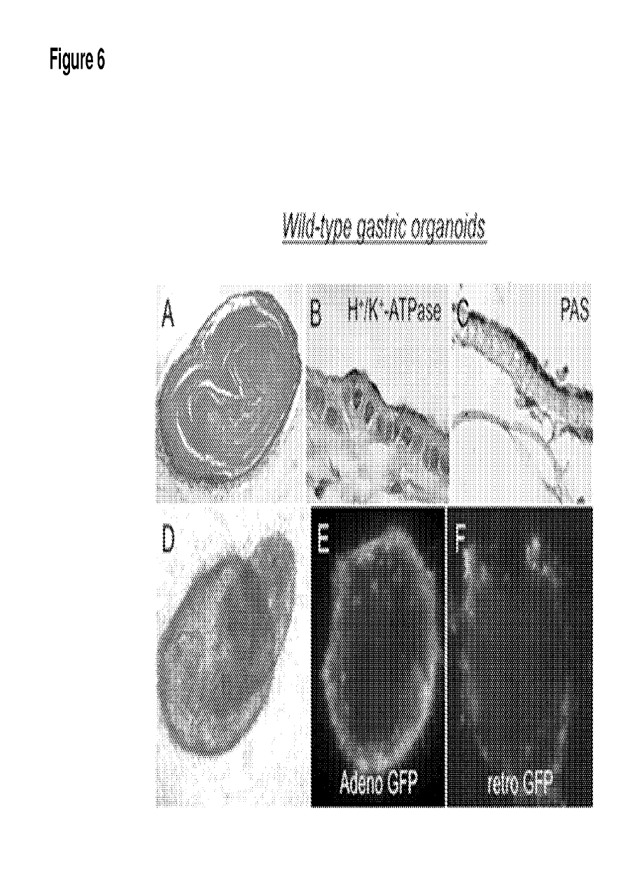

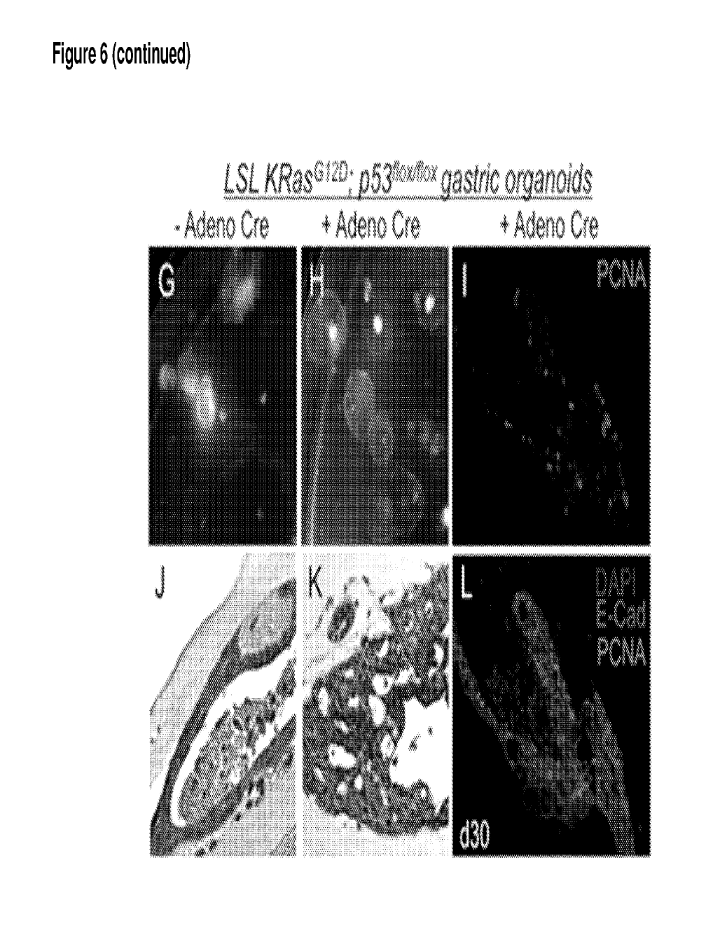

The conditions used above to culture colonic explants were applied without modification to gastric tissue. Air-liquid interface (ALI) gastric cultures were observed to grow as epithelial spheroids with multi-lineage differentiation (PAS, H⁺/K⁺ ATPase) (FIG. 6A-C).

Gastric organoids were robustly infected by adenovirus and retrovirus (FIG. 6D-F). Ad Cre-infected KRasG¹²D; p53^{flox/flox} (KP) gastric organoids are dysplastic, proliferative and invasive (FIG. 6G-L).

### Example 4

### Lung Alveolar Cultures

ALI has been previously used to culture primary mouse bronchioles by (a) initial growth on plastic, followed by secondary direct culture on a collagen-coated transwell (Yamaya, M., et al. (1992) Differentiated structure and function of cultures from human tracheal epithelium. Am J Physiol 262, L713-724; Widdicombe, et al. (2005) Expansion of cultures of human tracheal epithelium with maintenance of differentiated structure and function. Biotechniques 39, 249-255), or (b) with collagen gel/Matrigel co-culture with transformed fibroblasts (Delgado, O., et al. (2011) Multipotent capacity of immortalized human bronchial epithelial cells. PLoS One 6, e22023); robust alveolar culture has not been previously demonstrated.

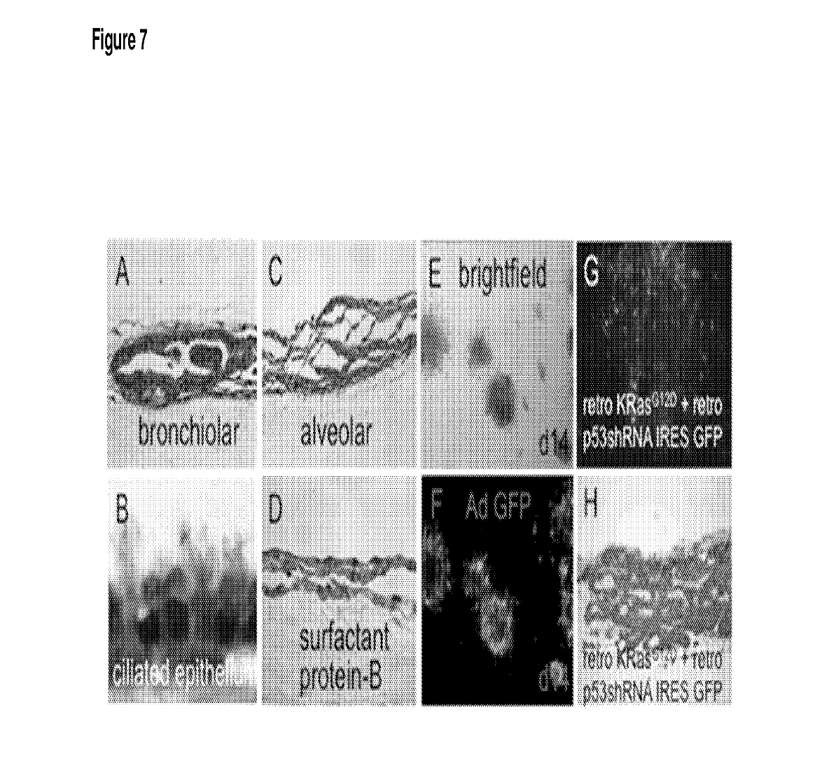

The ALI system was applied, again without modification, to the culturing and transformation of primary lung organoids. The colon and stomach ALI conditions in examples 1-3 promoted primary lung organoid culture for 4 weeks or more, i.e. in the absence of an initial growth on plastic or co-culture with transformed fibroblasts as disclosed in previous reports. Lung organoids possessed ciliated epithelium (FIGS. 7A-B) and regions of possible alveolar morphology expressing the type 2 pneumocyte marker surfactant protein B (SP-B) (FIGS. 7C-D).

Robust adeno/retro infection (FIGS. 7E-F, 10) inducing marked dysplasia with retro KRas^{G12D}+retro p53 shRNA IRES GFP infection.

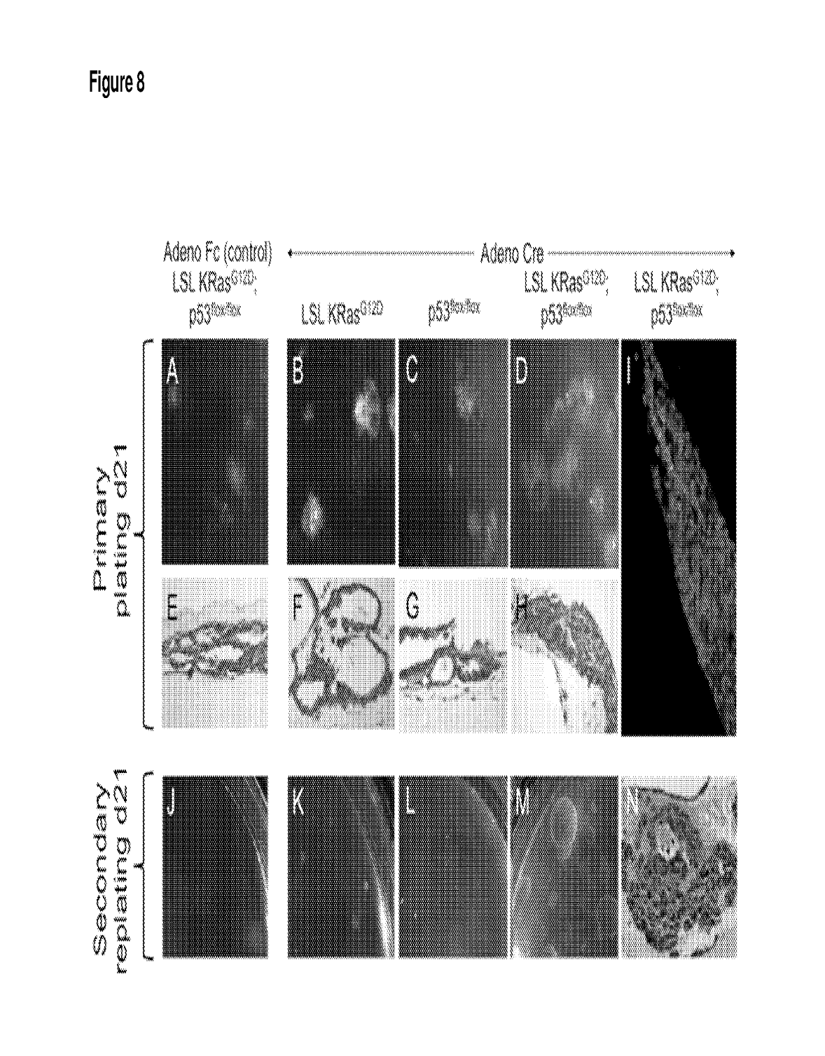

Lung organoids that are KRas^{G12D}, or p53-null or both by in vitro Ad-Cre infection of tissue from appropriate mouse strains were easily prepared (FIG. 8B-D). Ad Cre infection of KRas^{G12D}; p53^{flox/flox} (i.e. the “KP 2-gene module”) lung organoids (FIG. 8H) but not either KRas^{G12D} or p53^{flox/flox} (i.e. the “K 1-gene module” or “P 1-gene module”) lung organoids (FIG. 8F, G) induced pronounced dysplasia vs. wild-type (the “0-gene module”). The KP organoids exhibited both vigorous growth and a marked “polycystic” phenotype (FIG. 8D) with significant nuclear atypia/pleiomorphism, cellular stratification and significant mitoses consistent with adenocarcinoma by d30 of culture (FIG. 8H, I).

A serial replating assay for the transformed lung organoids was developed. Replated single cell suspensions from KRas^{G12D}; p53-null organoids grew much more vigorously (FIG. 8M, N) than wt, KRas^{G12D} or p53-null (FIG. 8JL) upon secondary passage, analogous to AKPS vs A colon. Thus, the synergistic effects of KRas^{G12D} and p53 in the 2 gene module was demonstrated by multiple criteria (dysplasia, polycystic morphology and serial replating).

Identical ALI methods to those used above have also been used successfully to culture and transform organoids from pancreas and bladder.

### Example 5

### Kidney Cultures

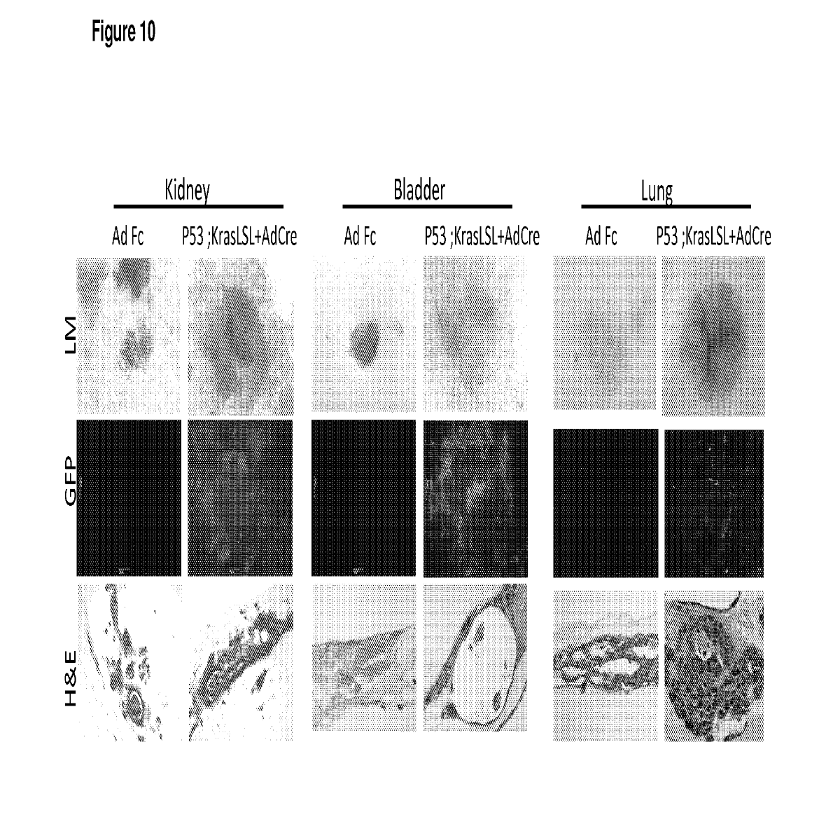

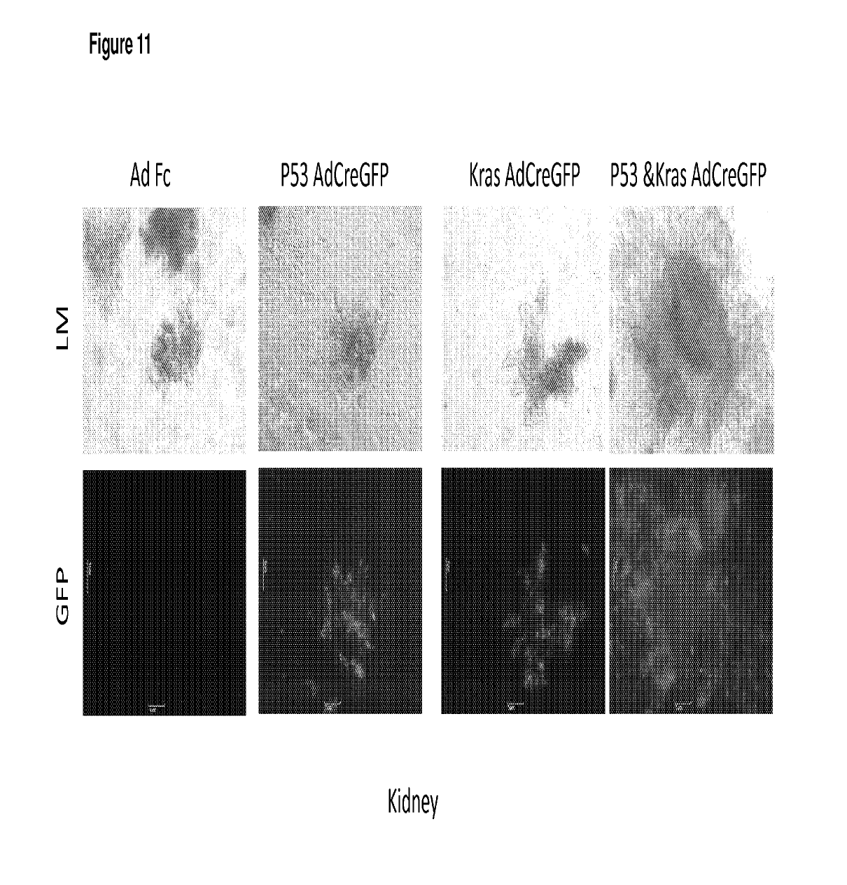

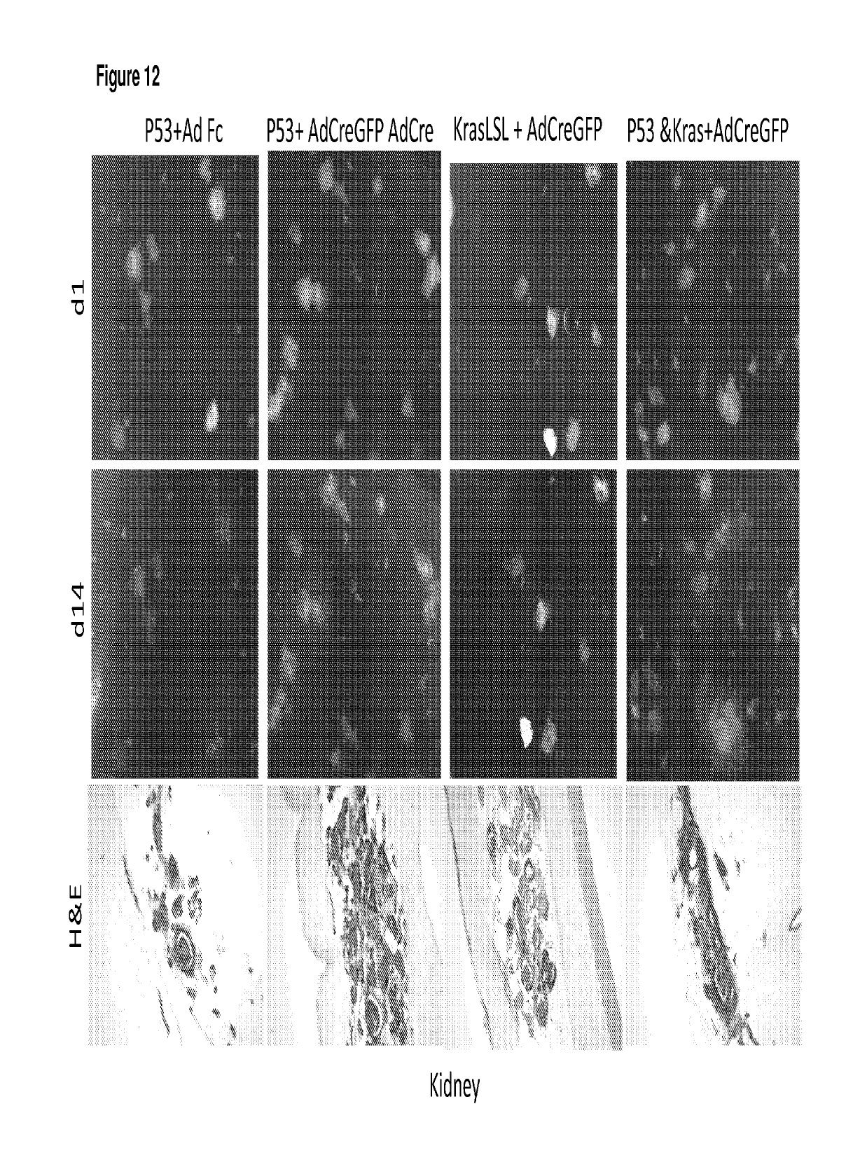

The ALI system was applied, again without modification, to the culturing and transformation of kidney tissue. Kidney organoids that are KRas^{G12D}, or p53-null or both by in vitro Ad-Cre infection of tissue from appropriate mouse strains were easily prepared (FIGS. 10-12).

### Example 6

### Bladder Cultures

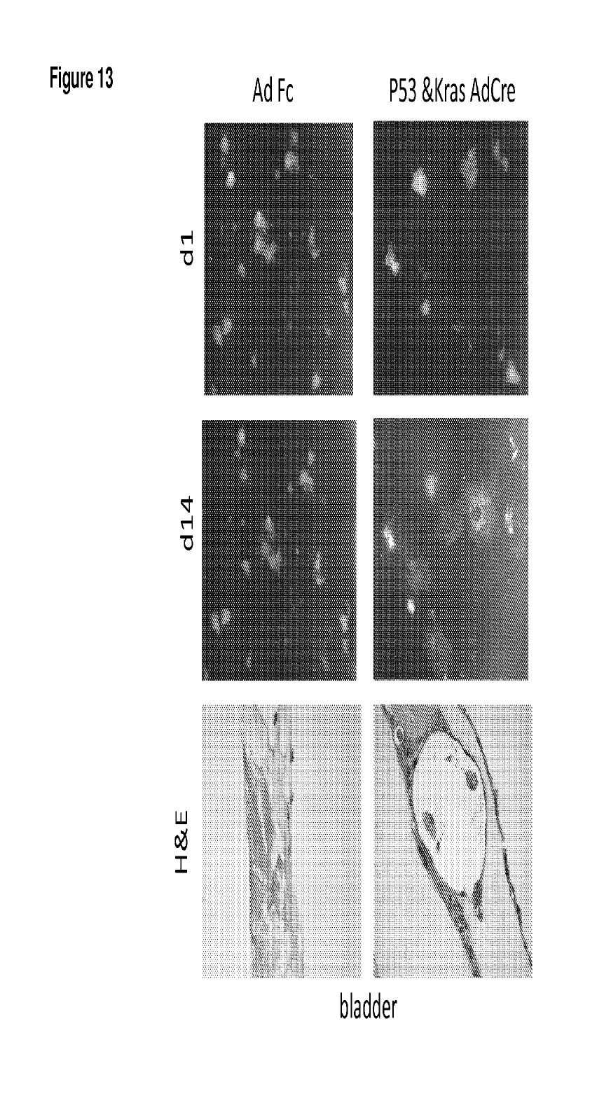

The ALI system was applied, again without modification, to the culturing and transformation of bladder tissue. Bladder organoids were prepared that were KRas^{G12D} and p53-null by in vitro Ad-Cre infection of tissue from appropriate mouse strains (FIGS. 10 and 13).

### Example 7

### Pancreatic Cultures

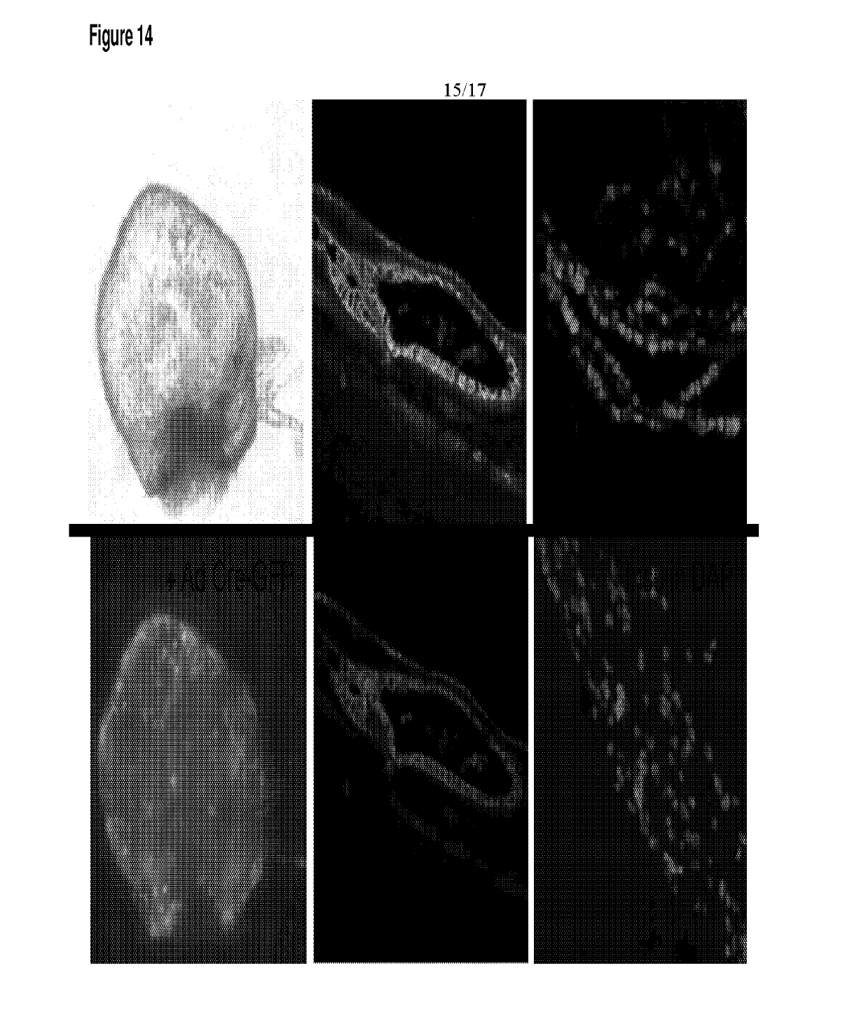

Primary mouse pancreatic organoid cultures were prepared using the ALI system. Brightfield images, GFP fluorescence after infection by adenovirus Cre-GFP, and immunofluorescence for the markers E-Cadherin, Pdx1, PCNA and insulin at day 10 demonstrate that these organoids grew well in culture (FIG. 14).

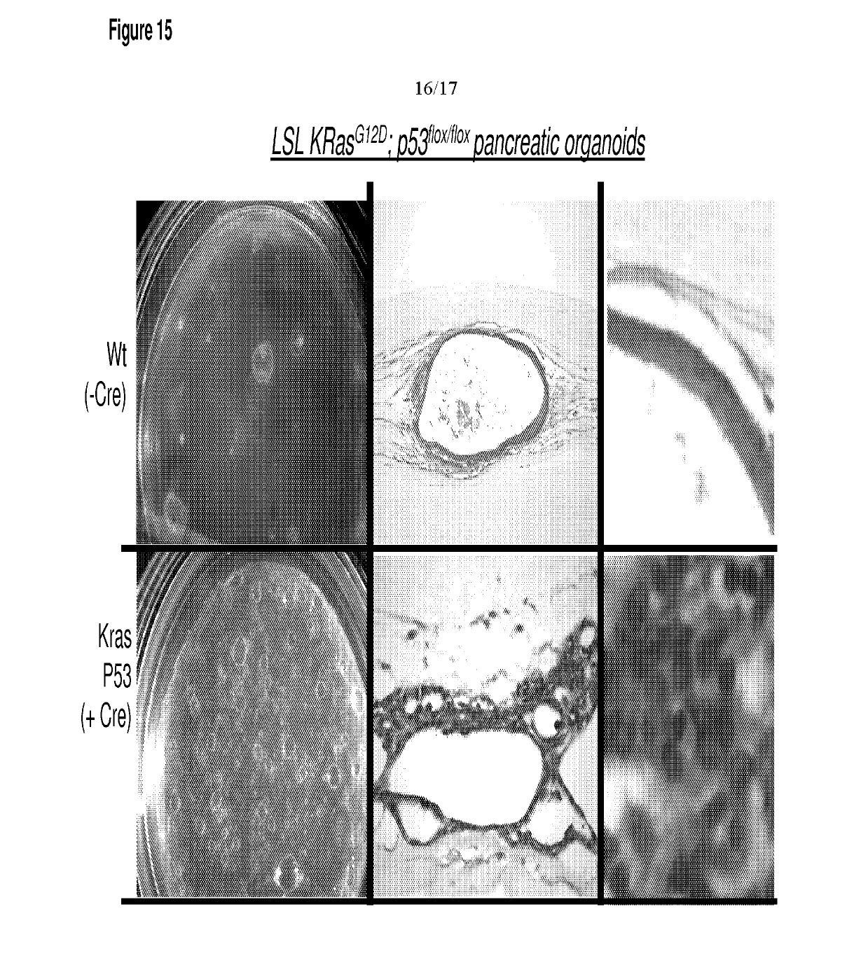

Pancreatic organoid culture from LSL KRas^{G12D}; p53^{flox/flox} mice were oncogenically transformed by adenovirus Cre-GFP infection. Cre-induced Kras activation and p53 deletion resulted in increased growth as well as histologic transformation (FIG. 15).

### Example 8

### High-Throughput Screening (HTS) Format

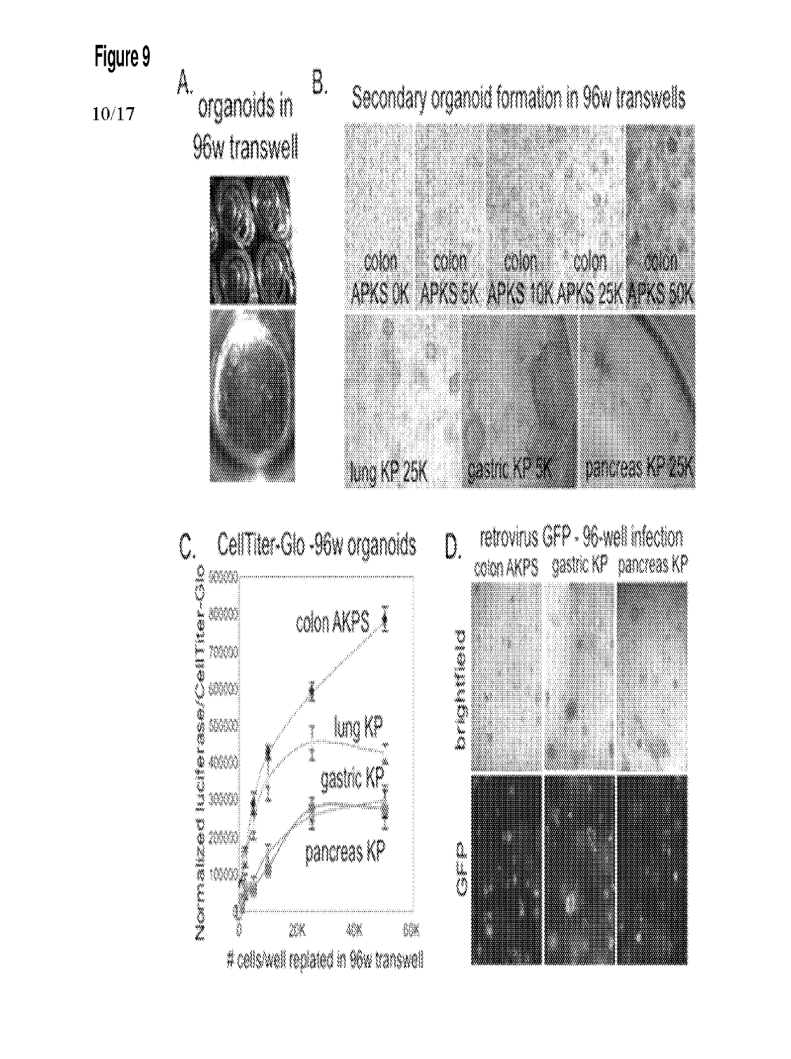

Several of the organoid ALI systems described herein have been used for high-throughput screening (HTS) by utilizing 96-well transwell inserts. Transformed organoids, e.g. colon AKPS, lung KP, gastric KP and pancreas KP organoids were disaggregated from standard single 35 mm transwells (c.f. FIG. 1A, FIGS. 4-8), and replated as single cell suspensions into ALI 96-well transwells in collagen gel (FIG. 9A). The replated cells for all organ systems formed secondary organoids in the 96 well transwells (FIG. 9AB) and exhibited highly reproducible and scalable growth, as quantitated in 96 well transwells over a dynamic range of detection from <1000 to ˜25000 cells (FIG. 9C). Further, we obtained extremely robust retrovirus GFP infection in 96 well transwells for all organ systems (FIG. 9D). These studies strongly support the utility of multiplexed organoid culture for functional gene validation and therapeutic screening applications.

### Example 9

### High-Throughput Functional Validation of Single TOGA Lung Adenocarcinoma (LUAD) Driver Genes Acting in Concert with KRas^{G12D} or p53

Primary organoids with oncogene manipulation (c.f. KRas, p53, APC) allow both positive selection and expansion of starting material which can be replated from single cell suspensions to generate secondary organoids in multiwell format, as exemplified by colon AKPS, and lung, stomach and pancreas KP (FIGS. 2, 5, 8-9). Secondary passage further allows highly accurate cell plating that is compatible with reproducible High-throughput (HT) measurement of proliferation over a broad dynamic range of cell numbers (FIG. 9C) while avoiding prolonged passage that is characteristic of established transformed cell lines (c.f. NIH60). Described here is the HT validation of putative TOGA LUAD individual driver loci and multigene modules in the context of either KRas^{G12D} or p53 loss (which comprises 20-30% and 70% of lung adenocarcinomas, respectively (Ding, L., et al. (2008) Somatic mutations affect key pathways in lung adenocarcinoma. Nature 455, 1069-1075; Greulich, H. (2010) The genomics of lung adenocarcinoma: opportunities for targeted therapies. Genes Cancer 1, 1200-1210; Kan, Z., et al. (2010) Diverse somatic mutation patterns and pathway alterations in human cancers. Nature 466, 869-873)), and define essential components of complex multigene modules.

TCGA LUAD Driver Loci.

Lung adenocarcinoma driver module contents are based on Ding et al. (Ding, L., et al. (2008) Somatic mutations affect key pathways in lung adenocarcinoma. Nature 455, 1069-1075) and additional fractional factorial (FF) analyses. This includes 2-gene modules containing loci co-mutated with KRas^{G12D} or p53 as prioritized for prevalence, driver probability and clinical relevance. Such loci that are co-mutated with KRas^{G12D} or p53 are candidates to exhibit transforming synergy with KRas^{G12D} or p53 and are systematically evaluated by the methods here for such activity.

Retroviruses for Lung Adenocarcinoma Tumor Suppressors.

Possible tumor suppressor mechanisms for candidate LUAD driver loci co-mutated with KRasG12D or p53 are: (1) inactivating mutation—genomic deletion, as in nonsense point mutations, out-of-frame small (<10 bp) insertions or deletions, splice-site changes and large (>10 bp) deletions or insertions, (2) gene conversion of a mutation in both alleles (3) biallelic inactivating mutations for a given gene, and (4) inactivating mutations in combination with transcriptional fold decrease from the wild type allele. For such loci, shRNA knockdown is performed using next-generation 29-mer whole genome murine shRNA clones in the retroviral pRFP-V-RS vector (Origene) when possible. Here, the U6 promoter drives both a puromycin marker and the shRNA cassette, and a CMV-RFP (or -GFP in pGFP-V-RS) element is also present for titering and for monitoring viral transduction of lung organoids. Multiple shRNA (3-5) are evaluated per target to minimize off-target effects and minimize false-negatives. p53 and luciferase shRNA (FIGS. 3,7) are used as positive and negative controls.

Retroviruses for Lung Adenocarcinoma Oncogenes.

Criteria for putative dominantly active oncogenes include (1) recurrent mutations, (2) genomic amplification and (3) transcriptional fold increase compared to matched normal tissue. Putative dominant oncogenes are modeled by retroviral cDNA overexpression via homologous recombination of full-length ORFeome clones (Open Biosystems) into a Gateway-adapted version of a retrovirus IRES puro/RFP vector. Particularly recurrent TCGA LUAD nonsynonymous mutations in which a functional consequence is not clear (i.e. not an INDEL), or mutations predicted to alter function (i.e. large mutations, deletions or stop codons) the cognate mutated allele are created by site-directed mutagenesis (QuikChange) to capture constitutively active or gain-of-function mutants, or publically available plasmids used (c.f. EML4-ALK, EGFR^{G719S}, EGFRL858R, EGFR (deli) L747-E749del, etc.)

ShRNA and cDNA retroviruses are generated in ecotropic Phoenix cells to restrict viral tropism to mouse tissues, avoiding safety issues with oncogene-expressing retro capable of infecting humans, followed by concentration by ultracentrifugation and FACS titering on NIH3T3 cells (GFP, RFP) yielding titers of >10⁸/ml; empty retrovirus are the negative control.

High-Throughput Organoid Culture and Retroviral Infection.

Lung organoids are generated from LSL KRas^{G12D} mice in ALI cultures exactly as described above. The lung is rapidly minced and resuspended in collagen I gel and plated into 35 mm Millicell-CM transwell culture inserts (Millipore, Mass.) on top of an acellular layer of collagen I, and placed in an outer 60 mm dish containing Ham's F-12/FCS. Adenovirus Cre-GFP is added to the culture medium at plating (c.f. FIG. 2, 6-9) to activate KRas^{G12D} expression. After 3d to allow deletion of the floxed LSL cassette, the resultant KRas^{G12D} organoids are disaggregated and FACS sorted to create a GFP+(surrogate for KRas^{G12D}) single cell suspension and passaged into 96-well ALI transwell culture at 50000 cells/well (FIG. 9) with each well having polybrene and a single ecotropic retrovirus encoding shRNA/cDNA in pRFP-V-RS (moi 100:1) representing a single locus to be tested for synergy with KRas^{G12D}. Note that this therefore generates a minimally passaged, 96-well arrayed isogenic series of KRas^{G12D} organoids differing only in the single retrovirus-manipulated drivers to be tested for transforming synergy. After 7d of infection, allowing for a moderate expansion, the 96-well arrayed organoids is disaggregated in situ, and a FACS Aria II sorter with automated cell deposition unit (ACDU) is used to split each isogenic sample at 5000 RFP+ (surrogate for retroviral infection) cells/well into new 96 well ALI (n=8 wells/module), followed by HT endpoint analysis after 7d for proliferation and invasion (see below). Identical procedures are followed for generation of p53^{flox/flox} organoids in 96-well format and for retroviral infection thereof to determine synergy with p53 loss.

From 10 neonatal KRas^{G12D} (or p53^{flox/flox}) mice or 1 adult mouse we routinely obtain 12×10⁶ cells from primary ALI lung organoids, sufficient for 3×96 well transwells at 50,000 cells/well and therefore for simultaneous retroviral engineering of up to 3×96=288 individual KRas^{G12D}-containing driver modules. Replating these organoids at n=8 into new transwells for HT measurement of proliferation and invasion scales the assay up to 3×8=(24) 96-well dishes.

Endpoint Analysis.

The KRas^{G12D} or p53 mutant isogenic organoids differing only in the test drivers, at n=8 wells/module in 96 well transwell, are assayed at day 7 post-plating for (a) proliferation as assessed by Cell Titer Glo (Promega) assay as mean+/−S.E. using a Molecular Devices AnalystGT 96-well plate reader in the Stanford High-Throughput Biosciences Core (HTBC) Facility (FIG. 9B); (b) invasion assessed by measuring migration of CellTracker Blue+ cells through the transwell (n=8) using a Molecular Devices AnalystGT 96-well plate reader, which is also conveniently capable of reading the bottom of transwells.

Loci passing the proliferation and/or invasion filters (above) are further assessed by histology using blinded evaluation of H&E and PCNA in larger 35 mm transwells (FIGS. 2, 4-8). The numerical dysplasia index (FIG. 4) sums nuclear grade, stratification, mitoses, invasion and extent of dysplasia. To filter false positives and control false negatives, shRNA knockdown by FACS/qPCR of RFP+EpCAM+ epithelium (FIG. 5) is documented and/or Western/IF are performed with appropriate mAb, use independent shRNA, and confirming cDNA overexpression. In some instances, loci passing the proliferation and/or invasion filters are also assessed for focus formation (FIG. 5EF) as a surrogate endpoint of oncogenic transformation. Promising loci may also be implanted subcutaneously into immunodeficient NSG mice (the mouse strains are currently of mixed background) (see FIG. 5GH for colon AKPS example), using tumor size and the histologic criteria above including dysplasia, nuclear pleiomorphism, mitoses and invasion.

### Example 10

### High-Throughput Analysis of Multigenic TCGA Lung Adenocarcinoma (LUAD) Driver Modules

Combinatorial gene manipulation via combined retroviral infection and floxed mouse alleles is a major asset of our organoid system. Accordingly, complex multigenic TGCA LUAD driver modules (up to 4 simultaneous events) are also assessed in primary organoids in HT format, allowing a substantial opportunity to define essential components and minimal modules, as relevant to driver dependency, “oncogene addiction” and therapeutic target identification.

Engineering of multigenic driver modules in lung organoids. Multigenic, expanded 4-component cancer driver modules are based on Ding et al. (Ding, L., et al. (2008) supra.) and additional fractional factorial (FF) analyses. This includes 4-gene modules containing loci co-mutated with KRas^{G12D} or p53 as prioritized for prevalence, driver probability and clinical relevance. Such loci that are co-mutated with KRas^{G12D} or p53 are candidates to exhibit transforming synergy with KRas^{G12D} or p53 and are systematically evaluated by the methods here for such activity.

LUAD multigene modules are modeled using a combination of retroviral infection and floxed mouse alleles (c.f. FIGS. 4-9) and/or deletion analysis. As above, appropriate compound floxed mouse backgrounds (c.f. LSL KRasG12D, p53flox/flox, LSL p53 point mutants or both) for different modules are infected with Ad Cre-GFP to activate latent/floxed alleles for 3d. Subsequently, single cell suspensions from these oncogene-activated organoids are replated at 25000 cells/well into 96-well transwells as in FIG. 9B, C and infected with combinations of RFP+ retroviruses (1-4 simultaneous retroviruses) (c.f. FIG. 9D) encoding the additional components of the combinatorial module to be evaluated. For example, using the KRas/p53/STK11/NF1 (KPSN) module, Ad Cre-treated LSL KRas^{G12D}; p53^{flox/flox} cultures are re-passaged into 96 well format with retrovirus STK11 shRNA and NF1 shRNA. As with the single TOGA LUAD driver studies above, these are re-passaged by FACS at 5000 cells/well (n=8 wells/module) into new 96-well ALI cultures. Endpoint analyses at d7 after replating includes proliferation and invasion through the transwell; promising modules scoring in proliferation/invasion undergo serial passage, focus formation and in vivo tumorigenicity analysis.

Deletion Analysis to Define a Minimal Co-Segregating Gene Module.

Synergy between a given locus and KRas^{G12D} or p53 loss indicates sufficiency for oncogenic transformation. To demonstrate necessity, each individual gene is systematically omitted from the multigene driver module in primary organoid culture in 96 well format. Further, to define a “minimal module” sufficient for transformation, systematic deletion of multiple genes from the cassette is evaluated for residual transforming activity, again in 96 well format. This definition of essential components and minimal modules within prevalent and clinically relevant TOGA modules is highly relevant to “oncogene addiction” and therapeutic target identification.

As in the analysis of TOGA LUAD single drivers, the ease of combinatorial retroviral infection is exploited in combination with appropriate floxed mouse starting material with our HT capacity to engineer hundreds of distinct modules simultaneously. Floxed mice for KRas^{G12D}, p53, both or others allow initiation of the majority of driver modules, and purely viral approaches are also used.

### Example 11

### High-Throughput Analysis of Multigenic TOGA Driver Modules For Other Tumor Types

HT functional validation of driver loci from other organ systems is used to explore other solid tumor types. The organoid culture method and gene validation method as described above for TOGA LUAD driver modules is also applied to the following types of tumors. In many cases the same basal driver modules (c.f. KRasG12D, p53) is applied:

1. Colon adenocarcinoma. Colon adenocarcinoma driver modules are based on the TOGA colon adenocarcinoma (COAD) dataset. As demonstrated in the examples above, the colon organoid system is extremely well characterized for multigenic engineering (FIGS. 3-4), adenoviral and retroviral infection (FIG. 3) and multiwell culture (FIG. 9). More complex modules than for lung (2-3 gene modules) are used as the basal module in secondary passage into multiwell format onto which additional loci are layered given the requirement for multiple hits in the colon system (FIG. 4); and starting material is readily available (c.f. APCflox/flox; LSL KRasG12D; p53^{flox/flox} mice, c.f. FIG. 2).

2. Rectal adenocarcinoma. Rectal adenocarcinoma driver modules are based on the TOGA rectal adenocarcinoma (READ) dataset. Rectal tissue is used in the organoid culture system described as above. The basal drivers (c.f. APC, KRas, p53) used in the colon cancer modeling are used in rectal adenocarcinoma modeling.

3. Gastric, pancreas, and bladder carcinomas. Gastric, pancreas, and bladder carcinomas driver modules are based on the TOGA gastric adenocarcinoma, pancreatic adenocarcinoma, and bladder adenocarcinoma datasets (STAD, PAAD, BLCA, respectively). As demonstrated in the example above, primary gastric tissue organoid culturing, transformation and HT adaptation (FIG. 6, 9) is well characterized. Pancreatic and bladder organoid cultures have also been developed using the methods described above.

### Example 12

### High-Throughput Applications of Primary Organoids for Drug Discovery

TGCA data sets and organoid cultures are combined for high-throughput screening (HTS) drug discovery applications. An isogenic series of primary cultures (c.f. colon, lung, stomach) are generated containing the most prevalent co-mutated TOGA gene modules for diverse tumor types in multi-well format.

Isogenic Organoids Varying in Driver Module Composition for Drug Discovery in Diverse Solid Tumor Types.

The primary organoid system described herein affords us an unusual opportunity to generate an isogenic series of primary transformed tissue from a variety of organ systems. These isogenic series is engineered for the most prevalent and clinically relevant TOGA driver modules in HT format. Agents are tested against a battery of isogenic modules in HT format with proliferation and invasion as primary endpoints. Agents are tested in concentration gradients (e.g. for small molecule agents, at 10⁻⁸ M to 10⁻⁴ M) to generate relative sensitivity curves against different gene modules for each lead. The identification of driver modules for which an agent is particularly effective (i.e. sensitivity at low concentrations) yields improved understanding of the cellular mechanisms underlying those tumors and allows highly focused clinical trials in patients with those driver module(s).

Pilot Investigations for HTS.

Our transformed organoids from a variety of organs can be assayed for proliferation in 96-well ALI transwell plate formats with broad dynamic range and strong reproducibility (FIG. 9). Here, we address the important question of how tumor cells differing in driver module compositions vary in their global therapeutic response to therapeutics, using isogenic colon organoids to template the workflow for other more clinically significant modules to be identified by the TOGA and potential chemical library screening.

As proof-of-principle, the 1-4 gene colon driver modules described in FIG. 4 are systematically assayed against the Biomol ICCB Known Bioactives and FDA-approved Drug Library (1120 small molecules including common chemotherapeutic agents) available at the Stanford High-Throughput Bioscience Center (HTBC) to generate a chemosensitivity fingerprint for our established series of colon organoids (AKPS, AKP, APS, AKS, AP, AS, KP). In addition, a library of approximately 20-50 small molecules specifically targeting common oncogenic modules (e.g. the Hh pathway inhibitor GDC-0449, the Wnt pathway inhibitor IWR-1, RTKI inhibitors (c.f. EGFR, FGFR, MET etc.) is also assessed.

The colon organoids are plated at 5000 cells/well in 96-well transwell ALI culture (FIG. 9) and treated with the compounds at seven doses spanning 310 nM to 20 μM. The cells are then cultured for an additional 48 h, and (1) viable cells quantitated by CellTiter-Glo and (2) invasive cells quantitated by CellTracker Blue (FIG. 9). Each step is fully automated using the HTBC's integrated Caliper Life Sciences Staccato System (see Equipment Section) adapted with a 96-pin tool for compound transfers. Cheminformatic database tools at the HTBC are used to calculate 1050 values for each compound in the organoid assay. These studies reveal how distinct oncogenic modules confer differential responses to pharmacological challenge, providing leads for personalized therapeutics development.

### Example 13

### Organoids of Human Tissues

Process Development for Human Organoid Culture and Safe Engineering of Oncogenic Modules.

The mouse systems have decided advantages of floxed alleles, tissue-specific Cre/CreER strains allowing compartment- and/or stem cell-specific deletion/activation, abundant starting material and ecotropic viruses—affording a significant safety factor when contemplating high-throughput validation of potentially oncogenic loci. The human system lacks mouse genetic tools and requires amphitropic viruses with attendant safety concerns. A method for culturing human organoids that overcomes these issues is described below.

Preparation of Human ALI Organoid Cultures.

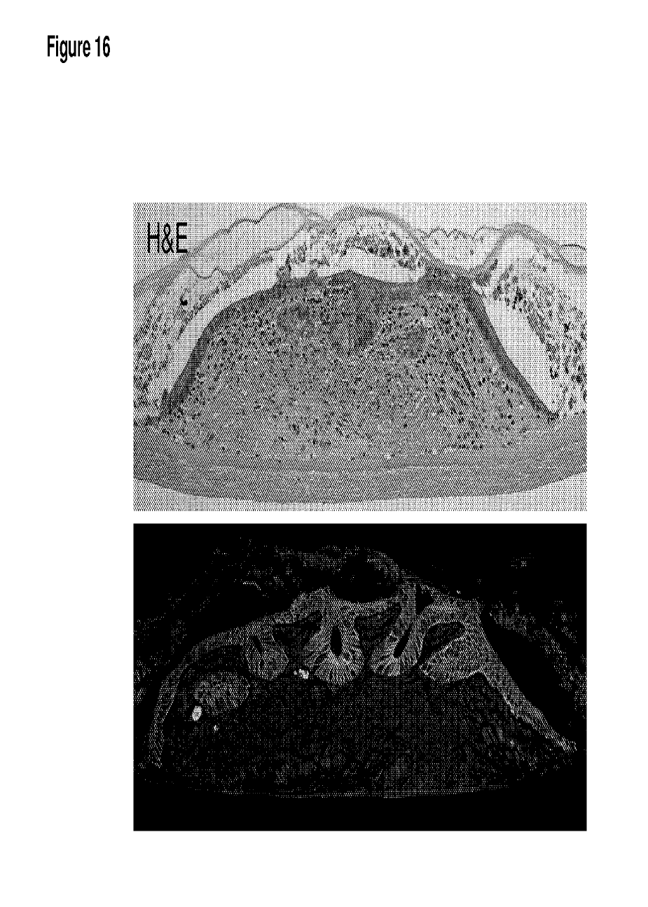

Human ALI organoid cultures may be prepared using the same air-liquid interface culture methods described in Example 1, above, without modification. For example, human colon organoids were prepared from human adult colon tissue using the air-liquid interface culture methods. These cultures resembled in vivo human colon tissue both structurally and by immunohistochemical markers (FIG. 16).

Human ALI Organoid Cultures Expressing Oncogenic Modules Via Ecotropic Lentivirus.

Organoids are cultured from adult human tissues as described above, and their growth rescued by transducing candidate TOGA drivers as rapidly as possible using ecotropic lentivirus. A non-integrating, replication-deficient adenovirus expressing Slc7a1, the host receptor for the envelope protein of ecotropic lentivirus and retrovirus is used to promote entry of ectotropic virus into human cells (Koch, P., et al. (2006) Transduction of human embryonic stem cells by ecotropic retroviral vectors. Nucleic Acids Res 34, e120; Takahashi, K., et al. (2007) Induction of pluripotent stem cells from adult human fibroblasts by defined factors. Cell 131, 861-872; Wang, H., et al. (1991) Cell-surface receptor for ecotropic murine retroviruses is a basic amino-acid transporter. Nature 352, 729-731). Adeno Slc7a1 infection strongly confers upon human 293T and HCT116 cells infectibility by ecotropic lentivirus GFP, which otherwise infects only mouse cell lines. Human organoids (c.f. lung, colon) are infected with adeno Slc7a1 or control adeno, followed immediately by infection by ecotropic (i.e. mouse-specific) lentiviruses expressing driver loci.

Human Organoid Cultures Derived from Human ES Cells.

Human ES cell-derived organoid cultures representing a variety of tissues are also employed. Intestinal tissue derived from hESCs by methods in the art (see, e.g. Spence, J R et al. (2011) Directed differentiation of human pluripotent stem cells into intestinal tissue in vitro. Nature 470(7332):105-9) is cultured under ALI conditions as disclosed herein for driver introduction.

What is claimed is:

1. A method for long term culture of mammalian organoids, comprising:
culturing mammalian tissue in a gel with an air-liquid interface,
wherein the culture provides for multilineage differentiation that maintains the ultrastructure and differentiation markers characteristic of the tissue.

2. The method according to claim 1, wherein the mammalian tissue is lung alveolar tissue.

3. The method according to claim 1, wherein the mammalian tissue is stomach tissue.

4. The method according to claim 1, wherein the mammalian tissue is pancreatic tissue.

5. The method according to claim 1, wherein the mammalian tissue is bladder tissue.

6. The method according to claim 1, wherein the mammalian tissue is liver tissue.

7. The method according to claim 1, wherein the mammalian tissue is kidney tissue.

8. The method according to claim 1, wherein cells of the organoids are viable for three months or more in culture.

9. The method according to claim 1, wherein the mammalian tissue is human tissue.

10. The method according to claim 1, wherein the cells of the tissue are experimentally modified prior to or during culture.

11. The method according to claim 10, wherein the cells are modified by introduction of a pathogen.

12. The method according to claim 10, wherein the cells are modified by introduction of a cancer driver.

13. An in vitro organoid culture derived by the method of claim 1.

14. A method for screening a candidate agent for an effect on a mammalian tissue, the method comprising:
contacting a candidate agent with an organoid culture according to claim 13, and determining the effect of the agent on the organoids in the culture.

15. The method according to claim 14, wherein the effect is tumorigenic.

16. The method according to claim 14, wherein the effect is anti-tumorigenic, and the cells of the organoids have been oncogenically transformed.

17. The method according to claim 14, wherein the effect is anti-viral or anti-bacterial, and the organoids have been infected with a virus or a bacteria.

18. A method for screening candidate cells for stem cell activity, the method comprising:
contacting candidate cells with an organoid culture according to claim 13, and determining the effect of the cells on the organoids.
